(19) United States
(12) Patent Application Publication
(10) Pub. No.: US 2023/0181659 A1
(43) Pub. Date: Jun. 15, 2023

(54) PHAGE-MEDIATED DELIVERY OF GENES TO GUT MICROBIOME

(71) Applicants: CZ BIOHUB SF, LLC, San Francisco, CA (US); THE REGENTS OF THE UNIVERSITY OF CALIFORNIA, Oakland, CA (US)

(72) Inventors: Kathy Lam, Oakland, CA (US); Peter Spanogiannopoulos, Oakland, CA (US); Peter Turnbaugh, Oakland, CA (US)

(21) Appl. No.: 17/923,865

(22) PCT Filed: May 13, 2021
(86) PCT No.: PCT/US2021/032182

§ 371 Date: Nov. 7, 2022

Related U.S. Application Data
(60) Provisional application No. 63/024,932, filed on May 14, 2020.

(51) Int. Cl. A61K 35/76; C12N 15/70; C12N 9/22; C12N 15/11; A61P 31/04; A61P 1/00; C12N 7/00

(52) U.S. Cl. CPC A61K 35/76; C12N 15/70; C12N 9/22; C12N 15/11; A61P 31/04; A61P 1/00; C12N 7/00; C12N 2310/20; C12N 2800/80; C12N 2795/14132; C12N 2795/14171

(57) ABSTRACT

The present disclosure provides materials and methods for selectively engineering at least one bacterial strain among a mixed population of bacterial strains in the gut of a subject. In some embodiments, a bacteriophage comprising at least one nucleic acid is administered which selectively infects a bacterial strain under conditions that allow expression of the nucleic acid. The present disclosure thus provides compositions and methods for precisely modifying or reducing a population of bacteria in a mixed population in the gut microbiome.

### CROSS-REFERENCE TO RELATED APPLICATIONS

The present application claims priority to U.S. Provisional Patent Application No. 63/024,932 filed May 14, 2020 the entirety of which is incorporated by reference herein.

### STATEMENT REGARDING FEDERALLY SPONSORED RESEARCH

This invention was made with government support under grant nos. R01 HL122593, R01 AT011117, and K08 AR073930 awarded by The National Institutes of Health. The government has certain rights in the invention.

### INCORPORATION BY REFERENCE OF MATERIAL SUBMITTED ELECTRONICALLY

The Sequence Listing, which is a part of the present disclosure, is submitted concurrently with the specification as a text file. The name of the text file containing the Sequence Listing is “55262_Seqlisting.txt”, which was created on May 13, 2021 and is 3,502 bytes in size. The subject matter of the Sequence Listing is incorporated herein in its entirety by reference.

### FIELD

The present disclosure relates generally relates to materials and methods for selectively engineering in vivo a bacterial strain, species, genus, etc., among a mixed population of bacteria in the gut of a subject.

### BACKGROUND

Broad interest in the influence that the gut microbiome has on host health and disease has inevitably led to the development of strategies with which to manipulate the structure and function of host-associated microbial communities. Various approaches for microbiome modification have recently been described, including engrafting bacterial strains in a naive host by providing exclusive nutrient sources in the diet (Shepherd, E. S., et al., Nature 557, 434-438 (2018); Kearney, S. M., et al., Cell Rep. 24, 1842-1851 (2018)) or treating with antibiotics (Staley, C. et al., Microbiome 5, 87 (2017), and Thompson, J. A., et al., Cell Rep. 10, 1861-1871 (2015)); introducing transient bacteria as a live therapeutic to complement an absent host metabolic activity (Isabella, V. M. et al., Nat. Biotechnol. 36, 857-864 (2018), and Kurtz, C. B. et al., Sci. Transl. Med. 11 (2019)); and chemically inhibiting microbial pathways active in host disease states (Zhu, W. et al., Nature 553, 208-211 (2018)). Bacteria have also been engineered to respond in vivo—that is, within the gut—to dietary compounds and synthetic inducers (Mimee, M., et al., Cell Systems 1, 62-71 (2015), and Lim, B., et al., Cell 169, 547-558.e15 (2017)), as well as to deliver genetic payloads to the gut microbiota in a conjugation-based strategy, opening the door to the simultaneous editing of diverse members of a bacterial consortium through delivery of a single donor organism (Ronda, C., et al., Nat. Methods 1 (2019)). Current strategies for microbiome editing, however, either lack species- or strain-level precision or require the introduction of an exogenous bacterium into the host.

Although bacterial viruses (bacteriophage or phage) have a long history of use in phage therapy (Merril, C. R., et al., Nat. Rev. Drug Discov. 2, 489-497 (2003); Abedon, S. T., Bacteriophage 1, 66-85 (2011); Lu, T. K. & Koeris, M. S., Curr. Opin. Microbiol. 14, 524-531 (2011); Bruussow, H., Expert Rev. Gastroenterol. Hepatol. 11, 785-788 (2017); and Abedon, S. T., et al., Front. Microbiol. 8, 981 (2017)), these applications have generally focused on the clearance of bacterial pathogens and made use of lytic phages in their native form. More recently, engineered phage carrying CRISPR-Cas constructs have been used in models of infection for sequence-specific targeting of enterohemorrhagic E. coli in larvae and antibiotic-resistant Staphylococcus aureus on animal skin (Citorik, R. J., et al., Nat. Biotechnol. 32, 1141-1145 (2014), and Bikard, D. et al., Nat. Biotechnol. 32, 1146-1150 (2014)). The sheer diversity of phages existing in nature and the ease with which they can be isolated against a wide range of bacteria make them attractive agents to engineer for gene delivery to bacterial cells colonizing the mammalian gut. Despite the huge potential of phages in this respect, there is currently a lack of in vivo models with which to study genetically tractable pairs of phages and their bacterial hosts, particularly in the context of genetic editing of an established host-associated microbial community.

Isolated nearly six decades ago from wastewater by a German scientist (Hofschneider, P. H., Zeitschrift fur Naturforschung B 18, 203-210 (1963)), M13 is a ssDNA filamentous phage belonging to the Inoviridae family in the ICTV classification of viruses Ackermann, H.-W., Methods Mol. Biol. 501, 127-140 (2009). It has an interesting life cycle in which it replicates and releases new virions from the cell without causing lysis (Salivar, W. O., et al., Virology 24, 359-371 (1964)). It is able to infect E. coli and related Enterobacteriaceae carrying the F sex factor that encodes proteins forming the conjugative F pilus (designated as F+, F′, or Hfr) (Lee, G. S. & Ames, G. F., J. Bacteriol. 159, 1000-1005 (1984), and Lin, A. et al. PLoS One 6, e19991 (2011)) where the pilus acts as the primary phage receptor and the inner membrane protein TolA as the co-receptor (Riechmann, L. & Holliger, P., Cell 90, 351-360 (1997), and Lubkowski, J., et al., Structure 7, 711-722 (1999)). M13 has made impressive contributions to the field of molecular biology—from the development of M13-based vectors for cloning, sequencing, and mutagenesis (Yanisch-Perron, C., et al., Gene 33, 103-119 (1985), Sanger, F., et al., J. Mol. Biol. 143, 161-178 (1980), and Zoller, M. J. & Smith, M., et al., Nucleic Acids Res. 10, 6487-6500 (1982) to its application in phage display (Smith, G. P. & Petrenko, V. A., Chem. Rev. 97, 391-410 (1997), and Sidhu, S. S., Biomol. Eng. 18, 57-63 (2001))—making it a very well characterized phage with excellent resources. In particular, the development of phagemid vectors that have both a plasmid origin of replication and an origin for packaging by M13 (e.g., ColE1 and f1, respectively) combine the advantages of plasmid DNA manipulation using standard techniques with the ability to easily package recombinant DNA into virions and generate phage preparations of high titer.

Phage M13 has been used previously in mice; for example, phage-displayed random peptide libraries have been screened in mice to identify \homing” peptides able to target organs or tumours (Pasqualini, R. & Ruoslahti, E., Nature 380, 364-366 (1996), Rajotte, D. et al., J. Clin. Invest. 102, 430-437 (1998), and Krag, D. N. et al., Cancer Chemother. Pharmacol. 50, 325-332 (2002)). M13 has also been applied by intraperitoneal injection as a bactericidal agent against E. coli by engineering it to deliver constructs that encode toxins lethal to the cell (Westwater, C. et al. Antimicrob. Agents Chemother. 47, 1301-1307 (2003)) or suppressors of the cellular response to DNA damage to enhance the efficacy of bactericidal antibiotics (Lu, T. K. & Collins, J. J., Proc. Natl. Acad. Sci. U.S.A. 106, 4629-4634 (2009)). Of relevance to the gut microbiome, M13 phage displaying antibody variable fragments against Helicobacter pylori surface antigens have been shown to reduce colonization by the bacterium in the mouse stomach when bacteria are pretreated with phage before oral inoculation (Cao, J. et al., Biochim. Biophys. Acta 1474, 107-113 (2000)). However, the use of M13 to deliver genetic constructs to established bacterial cells in the gastrointestinal tract for maintenance, rather than for bacterial killing or exclusion, has not been demonstrated.

### SUMMARY OF THE INVENTION

In various aspects, the present disclosure provides methods for selectively engineering at least one bacterial strain among a mixed population of bacterial strains in the gut of a subject comprising administering at least one bacteriophage comprising at least one nucleic acid, wherein said bacteriophage selectively infects the at least one bacterial strain under conditions that allow expression of said at least one nucleic acid.

In some embodiments, the at least one bacterial strain is a member of a species selected from the group consisting of Escherichia coli, Escherichia albertii, Klebsiella pneumoniae, and Salmonella typhimurium. In other embodiments, the at least one bacterial strain is a member of a genus selected from the group consisting of Escherichia, Salmonella, Shigella, and Klebsiella. In still another embodiment, the at least one bacterial strain is a member of Enterobacteriaceae.

The present disclosure also provides, in various embodiments, an aforementioned method wherein 1, 2, 3, 4, 5, 6, 7, 8, 9, 10 or more bacterial strains are selectively engineered by the same bacteriophage.

In one embodiment, the subject is a mammal. In another embodiment, the mammal is a human.

In various embodiments, an aforementioned method is provided wherein the at least one bacteriophage is selected from the group consisting of M13, T7, T4, lambda, T3, T1, P1 and Mu, and derivatives thereof. In another embodiment, 1, 2, 3, 4, 5 or more bacteriophage are administered.

In still other embodiment of the present disclosure, an aforementioned method is provided wherein the nucleic acid is a phagemid, a cosmid, and a phage-plasmid hybrid vector suitable for use with said at least one bacteriophage. In one embodiment, the nucleic acid comprises a phage-plasmid hybrid vector. In another embodiment, the phage-plasmid hybrid vector comprises one or more genes encoding a protein, an enzyme, or an RNA. In still another embodiment, the one or more genes is an antibiotic resistance gene. In yet another embodiment, the one or more genes encode an RNA-guided nuclease. In a related embodiment, the RNA-guided nuclease is Cas9. In still another embodiment, the Cas9 specifically targets an antibiotic resistance gene in the at least one bacterial species.

The present disclosure also provides, in various embodiments, an aforementioned method wherein the one or more genes encodes an enzyme selected from the group consisting of: an enzyme for drug activation, an enzyme for drug detoxification, an enzyme for transformation of dietary components into beneficial compounds for the host, an enzyme involved in one or more biosynthetic pathways for de novo production of beneficial compounds for the host (e.g. anti-inflammatory), an enzyme to aid long-term engraftment of the strain in the host gut, and an enzyme to increase competitive advantage of the strain in the host gut relative to the parent strain before gene delivery.

The present disclosure also provides other methods as provided below, each including the various embodiments described above.

In one embodiment, the present disclosure provides a method of treating a disease associated with at least one bacterial strain among a mixed population of bacterial strains in the gut of a subject comprising administering at least one bacteriophage to said subject comprising at least one nucleic acid, wherein said bacteriophage selectively infects the at least one bacterial strain under conditions that allow expression of said at least one nucleic acid, wherein said at least one nucleic acid comprises one or more genes.

In another embodiment, the present disclosure provides a method of increasing the growth of one or more target strain or species or genus in the gut microbiota of a subject comprising administering at least one bacteriophage to said subject comprising at least one nucleic acid, wherein said bacteriophage selectively infects the at least one bacterial strain under conditions that allow expression of said at least one nucleic acid, wherein said at least one nucleic acid comprises one or more genes.

In still another embodiment, the present disclosure provides a method of eliminating or reducing the population of one or more target strain or species or genus from an established community in the gut microbiota of a subject comprising administering at least one bacteriophage to said subject comprising at least one nucleic acid, wherein said bacteriophage selectively infects the at least one bacterial strain or species or genus under conditions that allow expression of said at least one nucleic acid, wherein said at least one nucleic acid comprises one or more genes.

In yet another embodiment, the present disclosure provides a method of conferring antibiotic resistance to one or more strain or species or genus of bacteria in an established community in the gut microbiota of a subject comprising administering at least one bacteriophage to said subject comprising at least one nucleic acid, wherein said bacteriophage selectively infects the at least one bacterial strain or species or genus under conditions that allow expression of said at least one nucleic acid, wherein said at least one nucleic acid comprises one or more genes that confer antibiotic resistance.

In another embodiment, the present disclosure provides a method of modifying the genome of one or more strain or species or genus of bacteria in an established community in the gut microbiota of a subject comprising administering at least one bacteriophage to said subject comprising at least one nucleic acid, wherein said bacteriophage selectively infects the at least one bacterial strain or species or genus under conditions that allow expression of said at least one nucleic acid, wherein said at least one nucleic acid comprises one or more genes.

In still another embodiment, the present disclosure provides a method of upregulating or downregulating at least one gene in one or more strain or species or genus of bacteria in an established community in the gut microbiota of a subject comprising administering at least one bacteriophage to said subject comprising at least one nucleic acid, wherein said bacteriophage selectively infects the at least one bacterial strain or species or genus under conditions that allow expression of said at least one nucleic acid, wherein said at least one nucleic acid comprises one or more genes or encodes for a functional RNA molecule capable of upregulating or downregulating the at least one gene.

In yet another embodiment, the present disclosure provides a method of selectively removing at least one bacterial strain or species or genus from a population of bacteria in the gut of a subject comprising administering at least one bacteriophage to said subject comprising at least one nucleic acid, wherein said bacteriophage selectively infects the at least one bacterial strain or species or genus under conditions that allow expression of said at least one nucleic acid, wherein said at least one nucleic acid comprises one or more genes.

In still another embodiment, an aforementioned method is provided wherein the bacteriophage is selected from the group consisting of M13, T7, T4, lambda, T3, T1, P1 and Mu, and derivatives thereof, and wherein said at least one nucleic acid encodes one or more sequences or genes involved in RNA-guided genome modification. In another embodiment, the bacteriophage is M13 and wherein said M13 comprises a nucleotide sequence encoding Cas9.

### BRIEF DESCRIPTION OF THE DRAWINGS

FIG. 1 shows that M13 bacteriophage can deliver a plasmid-borne antibiotic resistance gene to E. coli in the mouse gut. (FIG. 1a) A resistant subpopulation of E. coli can be selected in the gut when a β-lactam antibiotic is provided in the water. Streptomycin(Sm)-treated mice were gavaged with Sm^{R} MG1655 containing 99.9% ampicillin-sensitive (Amp^{S}) and 0.1% ampicillin-resistant (Amp^{R}) cells, and provided water containing Sm (n=5) or Sm+Amp (n=6). (FIG. 1b) A sensitive E. coli population is unable to maintain colonization in the gut when carbenicillin (Carb) is provided in the water. Mice were engrafted with either Sm^{R} MG1655 or Sm^{R} W1655 F+ (n=3 each) and Carb was provided in the water (shaded timepoints). (FIG. 1c) M13(pBluescript II) can infect F+ E. coli in the gut. Mice were split into three groups based on colonization and phage treatment: (1) Sm^{R} W1655 F− and live phage (n=3); (2) Sm^{R} W1655 F+ and heat-inactivated phage (n=3); (3) Sm^{R} W1655 F+ and live phage (n=4). 10¹⁴ phage were dosed and Carb was provided in the water. Total E. coli (black) and % Carb resistant colonies (Carb^{R}; gray) in mouse fecal pellets are shown. (d) M13-based delivery of an antibiotic resistance gene is dose-dependent. Varying doses (10⁷-10¹⁴) of M13(pBluescript II) were given to mice (n=2-3/dose) and Carb was provided in the water. After 2 days, Carb^{R} CFU/gram feces was determined. Dashed line indicates limit of detection.

FIG. 2 shows that M13-mediated delivery of CRISPR-Cas9 to E. coli in vitro causes impaired colony growth and can induce chromosomal deletions that encompass the targeted gene. (FIG. 2a) GFP+ E. coli exhibit a sick colony morphology after infection with M13 phage carrying GFP-targeting CRISPR-Cas9. NT (non-targeting) or GFPT (GFP-targeting) M13 were used to infect Sm^{R} W1655 F+ sfgfp or Sm^{R} W1655 F+ mcherry as a control. Cells were infected, diluted, and spotted onto media with selection for the vector; f1A or f1B indicates version of vector. (FIG. 2b) CRISPR-Cas9 targeting the sfgfp gene can induce loss of fluorescence. Colonies arising from infection with NT-M13 or GFPT-M13 were subjected to several rounds of streak purification on selective media to ensure phenotypic homogeneity and clonality. The majority (11/16) of GFPT clones exhibited a loss of fluorescence. (FIG. 2c) Clones exhibiting loss of fluorescence either lack an sfgfp PCR amplicon or exhibit an amplicon of decreased size. Genomic DNA was isolated from streak-purified clones and PCR was used to determine whether the sfgfp gene was present; PCR for the 16S rRNA gene was performed as a positive control. (FIG. 2d) Genome sequencing results confirm that nonfluorescent clones have chromosomal deletions encompassing the targeted gene. Read depth surrounding sfgfp locus for G9 clone (top, dark line, fluorescent control) and all nonfluorescent clones (grey lines). Deletion size indicated in text; range indicates a deletion flanked by repetitive sequences. Black arrow and vertical line denote position of targeting. Carb, carbenicillin.

FIG. 3 shows that M13-delivered CRISPR-Cas9 for sequence-specific targeting of E. coli in in vitro co-cultures of fluorescently marked isogenic strains. (FIG. 3a) M13-delivered GFP-targeting CRISPR-Cas9 leads to reduced competitive fitness of the GFP-marked strain. A co-culture of Sm^{R} F+ sfgfp and Sm^{R} F+ mcherry was incubated with NT-M13 or GFPT-M13 at a starting MOI of ˜500. Carbenicillin (Carb) was added to a final concentration of 100 μg/μl to select for phage infection. Co-cultures were sampled every 4 hours over 24 hours; cells were washed, serially diluted, and spotted onto non-selective media to assess targeting of the GFP-marked strain. (FIG. 3b) Carbenicillin in culture supernatants was not detectable within 4 hours of growth, using a carbenicillin bioassay against indicator strain Bacillus subtilis 168; bioassay detection limit approximately 2.5 μg/μl. (FIG. 3c) Flow cytometry of co-cultures 8 hours following the addition of phage and carbenicillin show reduced GFP+ events in the GFPT versus NT condition. Representative flow plot shows data from one of three replicates. Inset: bar graph quantifying percent GFP+ and mCherry+ events for three replicates (left); plating results for a single replicate on non-selective media (right). (FIG. 3d) GFPT CRISPR-Cas9 changes the shape of the distribution of GFP+ population. Histogram of mCherry+ and GFP+ events by intensity shows that a proportion of GFP+ cells in the GFPT condition have shifted to a state of lower fluorescence. Bars indicate the mean of three replicates; connected points are individual replicates.

FIG. 4 shows M13-delivered CRISPR-Cas9 for sequence-specific depletion of E. coli in the gut of mice colonized by competing fluorescently marked isogenic strains. (FIG. 4a) Timeline: Day −3, engraft with 50/50 mixture of streptomycin(Sm)-resistant Sm^{R} F+ sfgfp and Sm^{R} F+ mcherry; Day 0, dose with 10¹¹ NT-M13 or GFPT-M13 (n=10/group) and provide carbenicillin (Carb) in the water; Day 7, remove carbenicillin. (FIG. 4b) GFPT-M13 can lead to loss of the GFP-marked strain. Time series flow plots of fecal samples for one mouse from each of NT and GFPT groups. Top right: number of total, mCherry+, and GFP+ events. (FIG. 4c) Mice in GFPT group exhibited a decrease in number of fecal GFP+ events in over time compared to NT group; timepoints were excluded if both GFP+ and mCherry+ events were below background thresholds. Line graph: points indicate median; vertical lines, range. (FIG. 4d) Mice in GFPT group exhibited depletion or loss of the GFP-marked strain. Percent GFP+ and mCherry+ events for each mouse on Day 14. Mice were excluded if both GFP+ and mCherry+ events were both below background thresholds (final n=9 GFPT and n=10 NT). (FIG. 4e) A significant difference was observed in the percent of GFP+ events in fecal samples at Day 14 in the GFPT group compared to NT. Bars are medians; p-value, Mann-Whitney test.

FIG. 5 shows M13-delivered CRISPR-Cas9 can induce chromosomal deletions encompassing the targeted gene in E. coli colonizing the mouse gut. (FIG. 5a) Timeline: Day −5, engraft with double-marked streptomycin(Sm)-resistant Sm^{R} F+ sfgfp mcherry; Day 0, dose with 10¹¹ NT-M13 or GFPT-M13 (n=10/group) and provide carbenicillin (Carb) in the water; Day 7, remove carbenicillin. (FIG. 5b) GFPT-M13 can cause loss of GFP fluorescence in double-marked E. coli. Time series flow plots of fecal samples for select mice, one from each of NT and GFPT groups. Top right: total number of events; bottom right: mCherry+ events and GFP+ mCherry+ events. (FIG. 5c) Day 14 fecal samples of three mice in GFPT group exhibited mCherry-only fluorescence. Percent GFP+ mCherry+ and GFP− mCherry+ events for each mouse; mice were excluded if both populations were below the background threshold (final n=8/group). Dashed line indicates maximum mCherry fluorescence for NT group. (FIG. 5d) Colonies arising from culture of Mouse 18 Day 14 fecal sample confirmed presence of red-only fluorescence. (FIG. 5e) Genome sequencing results confirm red fluorescent isolates from Mouse 13, 14, and 18 have chromosomal deletions encompassing the targeted gene. Read depth surrounding sfgfp locus for GFP+ mCherry+ (Y1; light gray lines) and GFP− mCherry+ fluorescent (R1 or R2; dark lines) isolates from Day 2 fecal samples. Deletion size indicated in red; range indicates a deletion flanked by repetitive sequences. Black arrow and vertical line denote position of targeting.

### DETAILED DESCRIPTION

The recognition that the gut microbiome has a profound influence on human health and disease has spurred efforts to manipulate gut microbial community structure and function. Though various strategies have been described for modifying the gut microbiota, methods for phage-based genetic manipulation of resident members of the gut microbiota in vivo are currently lacking. The present disclosure provides, in various embodiments, that bacteriophage can be used as a vector for delivery of plasmid DNA to bacteria colonizing the gastrointestinal tract, using, by way of example, phage M13 and E. coli engrafted in the gut microbiota of conventional mice. The results presented herein provide a well-controlled and adaptable platform for in vivo microbiome engineering using phage and for the establishment of phage-based tools for a broader panel of human gut bacteria.

US Publication No. 2017/0246221, incorporated by reference herein in its entirety, discloses methods of inhibiting bacterial population growth and altering the relative ratio of sub-populations of bacteria in a mixed population. However, this publication fails to disclose use of specific phages such as phage M13 as chassis for delivery of plasmids, phagemids or other nucleic acids. Also, this publication focuses on inhibition of bacteria and altering relative ratios but does not mention genetic modification with RNA-guided nucleases to delete chromosomal genes but allow existence of the bacterium.

International Publication No. WO/2015/116531, incorporated by reference herein in its entirety, discloses compositions and methods to produce therapeutic bacteria phages capable of delivering nucleic acids to bacteria, modified phages and the use of the modified phages to deliver nucleic acids to bacteria. However, this publication fails to disclose in vivo data using phage to deliver RNA-guided nucleases for modifying bacteria in the gastrointestinal tract.

In various aspects, a method of altering bacterial abundance of microbiota in digestive organs of a subject in need thereof are provided, comprising administering to the subject a composition comprising at least one bacteriophage. In some embodiments, the digestive organs include the gut, intestines and digestive track of the subject.

In one embodiment of the present disclosure, a method of selectively engineering at least one bacterial strain among a mixed population of bacterial strains in the gut of a subject comprising administering at least one bacteriophage comprising at least one nucleic acid, wherein said bacteriophage selectively infects the at least one bacterial strain under conditions that allow expression of said at least one nucleic acid is provided. As used herein, the term “selectively” refers to the ability to specifically infect a desired bacterial strain, species, genus, etc. The term “engineering,” “engineer” or “engineered” as used herein means modifying a bacterial cell, strain, species or genus, etc., by, for example, introducing a bacteriophage comprising a nucleic acid. Those of skill in the art will understand that this modification includes, for example, introduction of exogenous nucleic acid sequences into a bacterial genome or expression of an exogenous gene product (e.g., structural/functional nucleic acid or protein).

Established communities in the gut microbiota are difficult to manipulate in vivo. The present disclosure provides for the first time compositions and methods to manipulate, e.g., selectively engineer, established communities or community members by, for example, increasing the fitness of a target bacteria, reducing the fitness of a target bacteria, enhancing the growth of a target bacteria, reducing the growth of target bacteria, and eliminating a target bacteria.

In general, any bacterial cell that resides in the gut microbiota of a subject are contemplated herein. In various embodiments, exemplary bacterial strains include but are not limited to strains of the species Escherichia coli, Escherichia albertii, Klebsiella pneumoniae, and Salmonella typhimurium.

In various embodiments of the present disclosure, exemplary genus include but are not limited to Escherichia, Salmonella, Shigella, and Klebsiella.

In other embodiments, exemplary bacterial families include but are not limited to Enterobacteriaceae.

Phages or bacteriophages are viruses that infect bacteria. The use of phages for the treatment of bacterial infections (known as phage therapy) is known. For example, phages have been used in antibacterial therapy and biotechnology as antimicrobial targeting infectious agents for both medical and industrial purposes as well as for research in gene discovery and protein expression. In this regard, such phage therapy is the therapeutic use of bacteriophages to treat pathogenic bacterial infections. Such conventional phages have been used therapeutically to treat bacterial infections that do not respond to conventional antibiotic drugs. This treatment involves the infection of a pathogenic or targeted bacteria by the phage and destruction of the bacteria via the lytic cycle of the phage replication pathway, thus eliminating the bacteria.

Phages have also been utilized for research of various prokaryotic and eukaryotic systems and many of the basic concepts of modern molecular biology are a result of studying the genetics of phages. Because phages can accommodate the insertion of large amounts of heterologous nucleic acids, the phage is an ideal vehicle for the cloning and expression of transgenic material. Indeed, several industrial and biotechnical applications of phage are known. Primary applications in biotechnology include the use of bacteria phage for nucleic acid or genetic “library” screening, the generation of single stranded DNA for sequencing (a utility which has become obsolete with advances in DNA sequencing technologies) and phage display. Such conventional technologies rely on the ability of the recombinant phage to replicate and form infectious particles that can be amplified either on their own or with the assistance of a helper phage.

According to various embodiments, the present disclosure provides methods for selectively engineering at least one bacterial strain among a mixed population of bacterial strains in the gut of a subject comprising administering at least one bacteriophage. The phage or bacteriophage can be strain specific, species specific, genus specific and so on. Exemplary phages include but are not limited to M13, T7, T4, lambda, T3, T1, P1 and Mu, and derivatives thereof.

The phrase “at least one” with respect to bacterial strain or species or genus or family or bacteriophage or nucleic acid includes 1, 2, 3, 4, 5, 6, 7, 8, 9, 10 or more members of the specified class.

“Nucleic acids” that are carried by or otherwise transferred to a bacteria according to the disclosure can be, for example, a plasmid, a phagemid, a cosmid, and a phage-plasmid hybrid vector suitable for use with said at least one bacteriophage. Additionally, the nucleic acid can be a inear fragment of DNA that can be integrated in to the genome of a bacterium.

As will be appreciated by those in the art, the bacteriophage or nucleic acid may comprise one or more (e.g., 1, 2, 3, 4, 5, or more) genes encoding a protein, enzyme or RNA. Exemplary genes or gene products include, but are not limited to,
antibiotic resistance genes that confer resistance to beta-lactams, chloramphenicol, tetracyclines, streptomycin;
enzymes for drug activation or re-activation (e.g., beta-glucuronidase, tyrosine decarboxylase);
enzymes for drug inactivation or detoxification (e.g., tyrosine decarboxylase cardiac glycoside reductase, azo reductase);
enzymes for transformation of dietary components into beneficial compounds for the host;
enzymes involved in one or more biosynthetic pathways for de novo production of beneficial compounds for the host (e.g., anti-inflammatory molecules;
enzymes to aid long-term engraftment of the strain in the host gut (e.g., pathways allowing exclusive catabolism of specific nutrients provided in the diet;
enzymes to increase competitive advantage of the strain in the host gut relative to the parent strain before gene delivery;
RNA molecules; and
RNA-guided nucleases such as Cas9.

Numerous other methods are provided herein, including methods of selectively removing a bacterial strain from a population of bacteria in the gut of a subject, and methods of treating toxicity or reduced efficacy of drugs that are transformed by bacteria in the gut.

As used herein, the term “microbiome” refers to the community (e.g., “established community”) of organisms and genetic material of all microbes—bacteria, fungi, protozoa and viruses—that live on and inside the human body. The bacteria in the microbiome help digest our food, regulate our immune system, protect against other bacteria that cause disease, and produce vitamins, including B vitamins B12, thiamine and riboflavin, and vitamin K, which is needed for blood coagulation. As used herein, then, the term “gut microbiome” refers to the microorganisms and genetic material therein that live in the digestive organs which include the gut, intestines and digestive track.

The microbiome is essential for human development, immunity and nutrition. The bacteria living in and on us are not invaders but beneficial colonizers. Autoimmune diseases such as diabetes, rheumatoid arthritis, muscular dystrophy, multiple sclerosis, and fibromyalgia are associated with dysfunction in the microbiome. Disease-causing microbes accumulate over time, changing gene activity and metabolic processes and resulting in an abnormal immune response against substances and tissues normally present in the body. Autoimmune diseases appear to be passed in families not by DNA inheritance but by inheriting the family's microbiome. A person's microbiome may influence their susceptibility to infectious diseases and contribute to chronic illnesses of the gastrointestinal system like Crohn's disease and irritable bowel syndrome.

Each individual is provided with a unique gut microbiota profile that plays many specific functions in host nutrient metabolism, maintenance of structural integrity of the gut mucosal barrier, immunomodulation, and protection against pathogens. Gut microbiota are composed of different bacteria species taxonomically classified by genus, family, order, and phyla. Each human's gut microbiota are shaped in early life as their composition depends on infant transitions (birth gestational date, type of delivery, methods of milk feeding, weaning period) and external factors such as antibiotic use. These personal and healthy core native microbiota remain relatively stable in adulthood but differ between individuals due to enterotypes, body mass index (BMI) level, exercise frequency, lifestyle, and cultural and dietary habits. Accordingly, there is not a unique optimal gut microbiota composition since it is different for each individual. However, a healthy host—microorganism balance must be respected in order to optimally perform metabolic and immune functions and prevent disease development. Dysbiosis of gut microbiota is associated not only with intestinal disorders but also with numerous extra-intestinal diseases such as metabolic and neurological disorders. Understanding the cause or consequence of these gut microbiota balances in health and disease and how to maintain or restore a healthy gut microbiota composition should be useful in developing promising therapeutic interventions.

In various embodiments, the methods described herein can be used to treat a disease or alleviate the symptoms in a mammal, e.g, a human, man or woman, or male child or female child, or a human infant (e.g., no more than 1, 2, 3 or 4 years of age). Samples from such subjects may be obtained using a variety of techniques known in the art including, but limited to, collection of fecal sample, a biopsy, and a noninvasive capsule endoscopy sample.

As discussed in Rinninella et al., (Rinninella, E., et al., Microorganisms, 7(1):14 (2019); incorporated by reference in its entirety herein), gut microbiota are composed of several species of microorganisms, including bacteria, yeast, and viruses. Taxonomically, bacteria are classified according to phyla, classes, orders, families, genera, and species. Only a few phyla are represented, accounting for more than 160 species. The dominant gut microbial phyla are Firmicutes, Bacteroidetes, Actinobacteria, Proteobacteria, Fusobacteria, and Verrucomicrobia, with the two phyla Firmicutes and Bacteroidetes representing 90% of gut microbiota. The Firmicutes phylum is composed of more than 200 different genera such as Lactobacillus, Bacillus, Clostridium, Enterococcus, and Ruminicoccus. Clostridium genera represent 95% of the Firmicutes phyla. Bacteroidetes consists of predominant genera such as Bacteroides and Prevotella. The Actinobacteria phylum is proportionally less abundant and mainly represented by the Bifidobacterium genus.

As used herein, the term “phyla” or “phylum” refer to the major lineages of the domain Bacteria. As described herein, abundances of various taxonomic levels are increased or decreased in response to the methods described herein including phylum, class, order, family, genus and species. Exemplary members of each group are provided herein:

As used herein, the phrase “comparing the abundance” refers to comparing the presence of and amount of a bacterial species or strain relative to a baseline or threshold amount or relative to a “before and after” scenario where the amount is measured before and after a specified treatment.

Bacteriophage may be administered in various dosages, according to the present disclosure including, but not limited to, up to 10¹⁴ viral particles.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “and,” and “the” include plural referents unless the context clearly dictates otherwise. Thus, for example, reference to “a conformation switching probe” includes a plurality of such conformation switching probes and reference to “the microfluidic device” includes reference to one or more microfluidic devices and equivalents thereof known to those skilled in the art, and so forth. It is further noted that the claims may be drafted to exclude any element, e.g., any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. This is intended to provide support for all such combinations.

The following materials and methods were used in in the Examples described herein.

Strains, Plasmids, Phage, and Oligonucleotides.

Bacterial strains, plasmids, and phage used in this study, including descriptions and sources, are provided in Table 1.

TABLE 1

| | | | | |
|---|---|---|---|---|
| | | Reference | | |
| Resource | Relevant Characteristics | or Source | KL ID | Accession |

Strains

| | | | |
|---|---|---|---|
| XL1-Blue | Phage propagation with helper | Agilent | |
| MRF′ | M13KO7; TcR | | |
| DH5α | Routine cloning; phage | Bethesda | |
| | propagation with helper HP4_M13 | Research | |
| | | Laboratories, | |
| | | 1986 | |
| MG1655 | Derivative of K-12 | Bachmann, | |
| | | 1996 | |
| W1655 F− | Derivative of K-12; M13R | ATCC | |
| | | 23737 | |
| W1655 F+ | Derivative of K-12; M13S | ATCC | KL68 |
| | | 23590 | |
| MG1655 | Spontaneous rpsL-SmR | This study | KL52 |
| rpsL-SmR | (Lys42Arg) derivative of MG1655 | | |
| W1655 F− | Recombineered rpsL-SmR | This study | KL89 |
| rpsL-SmR | (Lys42Arg) derivative of W1655 F− | | |
| W1655 F+ | Recombineered rpsL-SmR | This study | KL90 |
| rpsL-SmR | (Lys42Arg) derivative of W1655 F+ | | |
| AV01::pAV01 | MG1655 with constitutive sfgfp | Vigouroux | |
| | clonetegrated at HK022 att site; KmR | et al., 2018 | |
| AV01::pAV02 | MG1655 with constitutive | Vigouroux | |
| | mcherry clonetegrated at lambda att site; | et al., 2018 | |
| | KmR | | |
| W1655 F+ | SmR W1655 F+ with sfgfp | This study | KL114 |
| rpsL-SmR sfgfp | transduced from AV01::pAV01; KmS | | |
| W1655 F+ | SmR W1655 F+ with mcherry | This study | KL115 |
| rpsL-SmR mcherry | transduced from AV01::pAV02; KmS | | |
| W1655 F+ | SmR W1655 F+ sfgfp with | This study | KL204 |
| rpsL-SmR sfgfp | mcherry transduced from AV01::pAV02; | | |
| mcherry | KmS | | |

Plasmids

| | | | | |
|---|---|---|---|---|
| pBluescript II | Commercial phagemid; CarbR | Agilent | | X52329.1 |
| KS(−) | | | | |
| pSIJ8 | Temperature-sensitive; lambda | Jensen et | | |
| | Red recombineering; CarbR | al., 2015 | | |
| pE-FLP | Temperature sensitive; | Addgene; | | |
| | constitutive flippase expression; CarbR | St-Pierre | | |
| | | et al., 2013 | | |
| pCas9 | Low-copy vector carrying cas9, | Addgene; | | |
| | tracrRNA, and CRISPR array; CmR | Jiang et al., | | |
| | | 2013 | | |
| pCas9-GFPT- | pCas9 with GFP-targeting | This study | pKL100 | |
| flA | spacer; fl-bla in orientation A; CmR | | | |
| | CarbR | | | |
| pCas9-GFPT- | pCas9 with GFP-targeting | This study | pKL101 | |
| flB | spacer; fl-bla in orientation B; CmR | | | |
| | CarbR | | | |
| pCas9-NT- | pCas9 with non-targeting spacer; | This study | pKL102 | |
| flA | fl-bla in orientation A; CmR CarbR | | | |
| pCas9-NT- | pCas9 with non-targeting spacer; | This study | pKL103 | |
| flB | fl-bla in orientation B; CmR CarbR | | | |

Phage or Helper

| | | |
|---|---|---|
| M13KO7 | Helper phage; KmR | NEB |
| VCSM13 | Helper phage; KmR | Agilent |
| HP4_M13 | Helper plasmid; KmR | Praetorius |
| | | et al., 2017 |
| P1 | Transducing phage | ATCC |
| | | 25404-B1 |

TcR, streptomycin-resistant; SmR, streptomycin-resistant; KmR, kanamycin-resistant; KmS, kanamycin-sensitive; M13R, M13-resistant; M13S, M13-sensitive; CarbR, carbenicillin-resistant; CmR, chloramphenicol-resistant

Oligonucleotides used in this study are provided in Table 2.

| | | SEQ | |
|---|---|---|---|
| Oligo | Sequence | ID | |
| ID | (5′-3′) | NO: | Purpose |
| PS-rpsLI | CGTGGCATGGAAATACTCCG | 1 | F primer to amplify rpsL for |
| | | | recombineering |
| PS-rpsL2 | GCATCGCCCTAAAATTCGGC | 2 | R primer to amplify rpsL for |
| | | | recombineering |
| PSP116 | AAACCCTTCACCTTCACCACG | 3 | Oligo 1 to generate GFPT |
| | AACAGAGAATTTG | | spacer |
| PSP117 | AAAACAAATTCTCTGTTCGTG | 4 | Oligo 2 to generate GFPT |
| | GTGAAGGTGAAGG | | spacer |
| PSP120 | AAACATCGCACATCCTGGTCG | 5 | Oligo 1 to generate |
| | CGACATTAAGAGT | | NT spacer |
| PSP121 | AAAAACTCTTAATGTCGCGAC | 6 | Oligo 2 to |
| | CAGGATGTGCGAT | | generate NT spacer |
| PSP108 | TTAATAAATGCAGTAATACAGG | 7 | Primer to sequence spacer in |
| | | | CRISPR array |
| KL215 | CCTGTCGACGGTATCGATAAG | 8 | F primer to clone |
| | CTTGATATCG | | fl-bla from |
| | | | pBluescript II as |
| | | | SalI fragment |
| KL216 | CCTGTCGACGATTATCAAA | 9 | R primer to clone fl-bla from |
| | AAGGATCTTCACCTAGATCC | | pBluescript II as SalI fragment |
| KL207 | CTGTTCACCGGTGTTGTTCC | 10 | F primer to amplify sfgfp |
| | | | fragment |
| KL200 | TTATTTGTAGAGTTCATCCATGCCG | 11 | R primer to amplify sfgfp |
| | | | fragment |
| BAC338F | ACTCCTACGGGAGGCAG | 12 | F primer to amplify 16S rRNA |
| | | | gene fragment |
| BAC805R | GACTACCAGGGTATCTAATCC | 13 | R primer to amplify 168 rRNA |
| | | | gene fragment |
| V4515F | TCGTCGGCAGCGTCAGATGTGTATAA | 14 | F primer for 16S rRNA gene |
| Nextera | GAGACAGGTGCCAGCMGCCGCGGTAA | | sequencing primary PCR |
| V4 806R | GTCTCGTGGGCTCGGAGATGTGTATA | 15 | R primer for 16S rRNA gene |
| Nextera | AGAGACAGGGACTACHVGGGTWTCTAAT | | sequencing primary PCR |
| Various | AATGATACGGCGACCACCGAGATCTAC | 16 | F primer for 16S rRNA gene |
| | ACNNNNNNNNTCGTCGGCAGCGTC | | sequencing indexing PCR |
| Various | CAAGCAGAAGACGGCATACGAGATNN | 17 | R primer for 16S rRNA gene |
| | NNNNNNGTCTCGTGGGCTCGG | | sequencing indexing PCR |

Minimum inhibitory concentration (MIC) assay. Cells were prepared by standardizing an overnight culture to an OD₆₀₀ of 0.1 using saline (0.85% NaCl), and further diluted ten-fold in saline then ten-fold in LB. The drug was prepared by dissolving the antibiotic in vehicle (sterile distilled water) and filter-sterilizing, then serially diluting two-fold in vehicle to prepare 100× stock solutions, and finally diluting ten-fold in LB for 10× stock. To wells of a 96-well plate, 60 μl of LB, 15 μl of drug, and 75 μl of cells were added and mixed well. Final drug concentrations ranged between 0.002 μg/ml to 1000 μg/ml for ampicillin and 0.24 μg/ml to 2000 μg/ml for carbenicillin. The plate was incubated overnight at 37° C. without shaking and OD₆₀₀ was measured the following morning after agitation.

16S rRNA gene sequencing. Mouse fecal pellets were stored at −80° C. DNA was extracted from single pellets using a ZymoBIOMICS 96 MagBead DNA Kit and 16S rRNA gene sequencing was performed using a dual indexing strategy (Gohl, D. M. et al., Nat. Biotechnol. 34, 942-949 (2016)). Briefly, a 22-cycle primary PCR was performed using KAPA HiFi Hot Start DNA polymerase (KAPA KK2502) and V4 515F/806R Nextera primers. The reaction was diluted in UltraPure DNase/RNase-free water (Life Tech 0977-023) and used as template for a 10-cycle secondary (indexing) PCR using sample-specific dual indexing primers. The reactions were normalized using a SequelPrep Normalization plate (Life Tech A10510-01) and the DNA was eluted and pooled. To purify and concentrate the DNA, 5 volumes of PB Buffer (Qiagen 28004) were added, mixed, and purified using a QIAquick PCR Purification Kit (Qiagen 28106). The DNA was gel extracted using a MinElute Gel Extraction Kit (Qiagen 28604), quantified by qPCR using a KAPA Library Quantification Kit for Illumina Platforms (KAPA KK4824), and paired-end sequenced on the Illumina MiSeq platform. Data were processed using a 16S rRNA gene analysis pipeline (https://github.com/jbisanz/AmpliconSeq) based on QIIME2 (Bolyen, E. et al., Nat. Biotechnol. 37, 852-857 (2019)) incorporating DADA2 (Callahan, B. J. et al., Nat. Methods, 13, 581-583 (2016).), and analyzed using R packages qiime2R v0.99.23 (https://github.com/jbisanz/qiime2R), phyloseq v1.33.0 (McMurdie, P. J. & Holmes, S., PLoS One, 8, e61217 (2013)), and phylosmith v1.0.4 (Smith, S. D., Journal of Open Source Software 4, 1442 (2019)).

Construction of streptomycin-resistant E. coli strains. Strains resistant to the antibiotic streptomycin were generated by either selection for spontaneous resistance or by lambda Red recombineering (Datsenko, K. a. & Wanner, B. L., Proc. Natl. Acad. Sci. U.S.A. 97, 6640-6645 (2000); and Jensen, S. I., et al., Sci. Rep., 5, 17874 (2015)). Spontaneous resistant mutants were selected by plating overnight cultures on LB supplemented with 500 μg/ml streptomycin. Lambda Red recombineering was later used to introduce a specific allele for genetic consistency between strains as different mutations in the rpsL gene can confer resistance to streptomycin (Timms, A. R., et al., Mol. Gen. Genet. 232, 89-96 (1992)). Briefly, cells were transformed with the Carb^{R} temperature-sensitive plasmid pSIJ8 (Jensen, S. I., et al., Sci. Rep., 5, 17874 (2015)), and electrocompetent cells were prepared from cells grown in LB carbenicillin at 30° C. to early exponential phase and lambda Red recombinase genes were induced by addition of L-arabinose to 7.5 mM. Cells were electroporated with an rpsL-Sm^{R} PCR product amplified from a spontaneous streptomycin-resistant mutant of MG1655 using primers PS-rpsL1 and PS-rpsL2, and recombinants were selected on LB supplemented with 500 μg/ml streptomycin. The pSIJ8 plasmid was cured by culturing in liquid at 37° C. in the absence of carbenicillin, plating for single colonies, and confirming Carb^{S}. The rpsL gene of Sm^{R} strains was confirmed by Sanger sequencing.

Construction of fluorescently marked E. coli strains. P1 lysates were generated of AV01::pAV01 and AV01::pAV02 carrying clonetegrated sfgfp and mcherry, respectively (Vigouroux, A., et al., Mol. Syst. Biol., 14, e7899 (2018)). Briefly, 150 μl of overnight culture in LB supplemented with 12.5 μg/ml kanamycin was mixed with 1 μl to 25 μl P1 phage (initially propagated from ATCC on MG1655). The mixture was incubated for 10 min at 30° C. to aid adsorption, added to 4 ml LB 0.7% agar, and overlaid on pre-warmed LB agar supplemented with 25 μg/ml kanamycin 10 mM MgSO₄. Plates were incubated overnight at 30° C., and phage were harvested by adding 5 ml SM buffer, incubating at room temperature for 10 min, and breaking and scraping off the top agar into a conical tube. Phage suspensions were centrifuged to pellet agar; the supernatant was passed through a 100 μm cell strainer, then through a 0.45 μm syringe filter, and lysates were stored at 4° C. For transduction, 1-2 ml of recipient overnight culture was pelleted and resuspended in 1/3 volume LB 10 mM MgSO₄ 5 mM CaCl₂). 100 μl of cells was mixed with 1 μl to 10 μl P1 lysate and incubated at 30° C. for 60 minutes. To minimize secondary infections, 200 μl 1 M sodium citrate was added, followed by 1 ml of LB. The mixture was incubated at 30° C. for 2 h, then plated on LB 10 mM sodium citrate 25 μg/ml kanamycin to select for transductants. For excision of the vector backbone including the kanamycin resistance gene and heat-inducible integrase, cells were electroporated with pE-FLP (St-Pierre, F. et al. ACS Synth. Biol. 2, 537-541 (2013)); transformants were selected on carbenicillin and confirmed for Km^{S}. pE-FLP was cured by culturing in liquid at 37° C. in the absence of carbenicillin, plating for single colonies, and confirming Carb^{S}. Strains were subsequently grown routinely at 37° C. For imaging fluorescent strains on agar, plates were typically incubated at 37° C. overnight, transferred to room temperature to allow fluorescence intensity to increase, and then imaged.

Mouse experiments with E. coli engraftment, antibiotic water, and phage treatment. Animal procedures were approved by the University of California, San Francisco (UCSF) Institutional Animal Care and Use Committee (IACUC), and animal experiments performed were in compliance with ethical regulations. Specific pathogen free female BALB/c mice from the vendor Taconic were used for all mouse experiments. Streptomycin water was prepared by dissolving USP grade streptomycin sulfate (VWR 0382) in autoclaved tap water to a final concentration of 5 mg/ml and passing through 0.45 μm filtration units. Mice were provided streptomycin water for 1 day, followed by oral gavage of 0.2 ml containing approximately 10⁹ CFU of streptomycin-resistant E. coli. Mice were kept on streptomycin water thereafter to maintain colonization. For selection with β-lactam antibiotics, USP grade ampicillin sodium salt (Teknova A9510) or USP grade carbenicillin disodium salt (Teknova C2110) was also dissolved in the water to a final concentration of 1 mg/ml; carbenicillin was preferred for its increased stability over ampicillin (Bobrowski, M. & Borowski, E. et al., Microbiology, 68, 263-272 (1971)). Drinking water containing streptomycin was prepared fresh weekly; with the addition of a β-lactam antibiotic, it was prepared fresh every 3-4 days. For phage treatment, filtered phage solutions stored at −80° C. were thawed and used directly for oral gavage. Unfiltered phage solutions were precipitated by diluting approximately 5-fold in PBS, adding 0.2 volumes phage precipitation solution (20% PEG-8000, 2.5 M NaCl), incubating for 15 min on ice, pelleting at 15,000-21,000 g for 15 min at 4° C., resuspending in PBS, centrifuging to pellet insoluble matter, and filtering through 0.45 μm. Heat-inactivated phage were prepared by incubating 1 ml aliquots at 95° C. in a water bath for 30 min. Streptomycin-treated mice colonized with Sm^{R} E. coli were orally gavaged with 0.2 ml of phage and placed on drinking water containing both streptomycin and carbenicillin.

Enumeration and culture of E. coli from mouse feces. Fecal pellets were collected from individual mice and CFU counts were performed on the same day to determine CFU per gram feces. Briefly, fecal samples (typically 10-40 mg) were weighed on an analytical balance and 250 μl to 500 μl PBS or saline was added. Samples were incubated for 5 min at room temperature and suspended by manual mixing and vortexing. Large particulate matter was pelleted by centrifuging at 100 g, ten-fold serial dilutions were made in PBS, and 5 μl of each dilution was spotted on Difco MacConkey agar (BD 212123) supplemented with the appropriate antibiotics, i.e., streptomycin (100 μg/ml) or carbenicillin (50 μg/ml). For qualitative assessment of the fluorescent strains in feces, samples were spotted onto LB supplemented with the appropriate antibiotics. For isolating E. coli from fecal samples for genomic or plasmid DNA analysis, the fecal suspension was streaked on agar, and single colonies were further streak-purified.

Construction of CRISPR-Cas9 phagemid vectors. Cultures were grown in LB or TB media supplemented with the appropriate antibiotics. Plasmid DNA was prepared by QIAprep Spin Miniprep Kit (Qiagen 27106), eluted in TE buffer, and incubated at 60° C. for 10 min. Samples were quantified using a NanoDrop One spectrophotometer. The vector pCas9 (Jiang, W., et al., Nat. Biotechnol. 31, 233-239 (2013)) was digested with BsaI (NEB R0535) and gel extracted with a QIAquick Gel Extraction Kit (Qiagen 28706). Spacers were generated by annealing and phosphorylating the two oligos (PSP116 and PSP117 for GFPT; PSP120 and PSP121 for NT (Jensen et al.) at 10 μM each in T4 ligation buffer (NEB B0202S) with T4 polynucleotide kinase (NEB M0201S) by incubating at 37° C. for 2 hours, 95° C. for 5 min, and ramping down to 20° C. at 5° C./min. The annealed product was diluted 1 in 200 in sterile distilled water and used for directional cloning by ligating (Thermo Scientific FEREL0011) to 60 ng of BsaI-digested, gel extracted pCas9 overnight at room temperature. Ligations were used to transform NEB 5-alpha competent cells (NEB C2987H) and the cloned spacer was verified by Sanger sequencing using primer PSP108. The trailing repeat was later confirmed to lack the starting 5′G, which did not interfere with GFP-targeting function. The 1.8-kb fragment carrying the f1 origin of replication and β-lactamase gene (f1-bla) was amplified from pBluescript II with SalI adapters using primers KL215 and KL216 and KOD Hot Start DNA polymerase (Millipore 71842-3). The PCR product was purified using a QIAquick PCR Purification Kit (Qiagen 28104), digested with SalI (Thermo Fisher FD0644), gel extracted, and used to ligate to SalI-digested, FastAP-dephosphorylated (Thermo Fisher FEREF0651) vector. Ligations were used to transform DH5α and clones were screened by restriction digest for both possible insert orientations (A or B) using XbaI (Thermo Scientific FD0684) and one of each orientation was saved for both the GFPT and NT phagemids.

Preparation of M13 carrying pBluescript II. This protocol was adapted from those to generate phage display libraries (Tonikian, R., et al., Nat. Protoc. 2, 1368-1386 (2007)). XL1-Blue MRF′ was transformed with pBluescript II (Agilent 212208). An overnight culture of this strain was prepared in 5 ml LB supplemented with tetracycline (5 μg/ml) and carbenicillin (50 μg/ml) and subcultured the following day 1-in-100 into 5 ml 2YT supplemented with the same antibiotics. At an OD₆₀₀ of 0.8, cells were infected with helper phage M13KO7 (NEB N0315S) or VCSM13 (Agilent 200251) at a multiplicity of infection of approximately 10-to-1 for 1 h at 37° C. The infected cells were used to seed 2YT supplemented with carbenicillin (100 μg/ml) and kanamycin (25 μg/ml) at 1-in-100, and the culture was grown overnight to produce phage. Cells were pelleted at 10,000 g for 15 min, and the supernatant containing phage was transferred. Phage were precipitated by adding 0.2 volumes phage precipitation solution, inverting to mix well, and incubating for 30 min on ice. Phage were pelleted at 15,000 g for 15 min at 4° C. and the supernatant was discarded. The phage pellet was resuspended in PBS at 1-4% of the culture volume. The resuspension was centrifuged to pellet insoluble material and transferred to a new tube. Glycerol was added to a final concentration of 10-15%. Phage preparations were aliquoted into cryovials and frozen at −80° C. for long-term storage.

Preparation of M13 carrying CRISPR-Cas9 phagemids. DH5α (HP4 M13) (Praetorius, F. et al., Nature 552, 84-87 (2017)) was transformed with the GFPT phagemid (pCas9-GFPT-f1A or pCas9-GFPT-f1B) or the NT phagemid (pCas9-GFPT-f1A or pCas9-GFPT-f1B) and plated on LB media containing carbenicillin and kanamycin. Transformants were inoculated into 5 ml 2YT supplemented with 100 μg/ml carbenicillin and 25 μg/ml kanamycin, incubated overnight, used 1-in-100 to seed 250 ml of the same media, and incubated overnight. Cells were pelleted at 10,000 g for 15 min, the supernatant was transferred to a new tube, 0.2 volumes of phage precipitation solution was added, and incubated 30 min on ice. Phage were pelleted at 20,000 g for 20 min with slow deceleration. The supernatant was completely removed, phage were resuspended in PBS at 1% of the culture volume, and glycerol was added to a final concentration of 10-15%. The phage solution was centrifuged at 21,000 g to pellet insoluble matter, filtered through 0.45 μm, and stored at −80° C.

Titration of M13 phage carrying phagemid DNA. Phage titer was determined using indicator strain XL1-Blue MRF′ or Sm^{R} W1655 F+. An overnight culture of the indicator strain in LB supplemented with the appropriate antibiotics was subcultured 1-in-100 or 1-in-200 into fresh media and grown to an OD₆₀₀ of 0.8. To estimate titer, serial ten-fold dilutions of the phage preparation were made in PBS, and 10 μl of each dilution was used to infect 90 μl of cells. After incubating at 37° C. for 30 min with shaking, 10 μl of the infection mix was spotted onto LB supplemented with carbenicillin. For more accurate titration, 100 μl of phage dilutions were mixed with 900 μl cells in culture tubes, incubated at 37° C. for 30 min with shaking, and 100 μl was plated on LB carbenicillin.

Enumeration of viable M13 from mouse feces. Mice were orally gavaged with 6×10¹³ M13(pBluescript II) or as negative controls, heat-inactivated phage or PBS. Approximately 100 mg of feces were collected at 0, 3, 6, 9, and 24 hours post-gavage, and samples at each timepoint were processed immediately. 500 μl PBS was added, samples were incubated for 5 min at room temperature, then suspended by manual mixing and vortexing. Samples were centrifuged at 21,000 g for 1 min, the supernatant was transferred to a new tube, and phage titer was determined against indicator strain XL1-Blue MRF′ by diluting samples in PBS, incubating with cells, and plating on LB supplemented with carbenicillin. For all dilutions and the undiluted suspension, 10 μl was used to infect 90 μl cells; additionally, for the undiluted suspension, 100 μl was used to infect 900 μl cells to maximize the limit of detection.

Assay for acid survival. Phage M13(pBluescript II) stored in PBS was diluted 1-in-100 in saline. Solutions varying in pH (1.2, 2, 3, 4, 5, 6, and 7) were prepared by mixing different ratios of 0.2 M sodium phosphate dibasic and 0.1 M citric acid. 200 μl of each pH solution was transferred to the wells of a microtiter plate, and 10 μl of phage was added containing 1×10⁹ M13(pBluescript II). Phage were incubated in the solution, and 10 μl was sampled at 5, 15, and 60 min. Samples were diluted 1-in-100 in PBS to make acidic samples neutral and phage titer was determined against indicator strain XL1-Blue MRF′ by plating on LB supplemented with carbenicillin. Solution-only controls were assayed simultaneously and cells were plated on LB to confirm viability of the indicator strain in the presence of samples originating from an acidic pH.

Targeting experiments in vitro with M13 CRISPR-Cas9. Overnight cultures of fluorescently marked Sm^{R} W1655 F+ sfgfp and mcherry were prepared in LB supplemented with streptomycin, subcultured 1 in 200 into fresh media, and grown to an OD₆₀₀ of 0.8. 900 μl cells (approximately 1×10⁹) was transferred to a culture tube, 100 μl phage (approximately 1×10¹⁰ for f1A vectors and approximately 5×10¹⁰ for f1B vectors) was added, and the tube was incubated at 37° C. for 30 min. The infection culture was transferred to a microfuge tube, cells were pelleted at 21,000 g for 1 min, and the supernatant was removed. Cells were washed twice by adding 1 ml PBS, vortexing, pelleting cells, and removing supernatant. Cells were resuspended in 1 ml PBS, and ten-fold serially diluted in PBS. 10 μl of each dilution was spotted onto LB supplemented with carbenicillin and 100 μl was plated on larger plates for isolating single colonies for analysis. Colonies were picked and streak-purified four times to ensure phenotypic homogeneity and clonality.

Co-culture experiments with sfgfp and mcherry-marked strains infected with M13 CRISPR-Cas9. Overnight cultures of fluorescently marked Sm^{R} W1655 F+ sfgfp and mcherry were prepared in LB supplemented with streptomycin. For each culture, three serial ten-fold dilutions were made in PBS, followed by a fourth ten-fold dilution into LB. Equal volumes of each were combined and 5 ml aliquots were transferred to culture tubes. Using a CFU assay, the input was determined to be 6×10⁶ CFU of each strain or 1×10⁷ CFU total. 10 μl (5×10⁹) M13 carrying CRISPR-Cas9 was added, the co-culture was incubated at 37° C. for 30 min, and carbenicillin was added to a final concentration of 100 μg/ml. The co-culture was sampled for the t=0 timepoint and then incubated for 24 hours with further sampling every 4 hours. At each timepoint, 200 μl was taken; 100 μl was used to assay carbenicillin in the media (see below) and the remaining 100 μl was used for plating as follows. To the 100 μl sample of culture, 900 μl was added and cells were washed by vortexing. Cells were pelleted by centrifuging at 21,000 g for 1 min, and 900 μl of the supernatant was removed. To remove residual phage and antibiotic, the wash was repeated once more by adding 900 μl PBS, vortexing, pelleting cells, and removing 900 μl. Cells were resuspended in the remaining 100 μl. Serial ten-fold dilutions were made in PBS and 10 μl of each dilution was spotted onto LB or LB carbenicillin.

Carbenicillin bioassay. Cultures were sampled over time, cells were pelleted at 21,000 g for 1 min, and the supernatant was transferred to a new tube and frozen at −20° C. until all timepoints were collected. The supernatants were thawed and assayed using a Kirby-Bauer disk diffusion test. An overnight culture of the indicator organism (Bacillus subtilis 168) was diluted in saline to an OD₆₀₀ of 0.1. A cotton swab was dipped into this dilution and spread across LB agar, antibiotic sensitivity disks (Fisher Scientific S70150A) were overlaid using tweezers, and 20 μl of the supernatant was applied to the disk. At the same time, carbenicillin standards were prepared from 1 μg/ml to 100 μg/ml and also applied to discs. Plates were incubated overnight at 37° C. and imaged the following morning.

Flow cytometry. For turbid in vitro cultures, samples were diluted 1-in-10,000 in PBS. For mouse fecal pellets, samples were used fresh or thawed from −80° C., and suspended in 500 μl PBS by manual mixing and vortexing. Fecal suspensions were incubated aerobically at 4° C. overnight to improve fluorescence signal. Samples were vortexed to mix, large particulate matter was pelleted by centrifuging at 100 g for 30 seconds, and the sample was diluted 1-in-100 in PBS. Samples were run on a BD LSRFortessa flow cytometer using a 530/30 nm filter for GFP fluorescence and 610/20 nm for mCherry fluorescence, with the following voltages: 750 V for FSC, 400 V for SSC, 700 V for mCherry, and 700-800 V (in vivo) or 650 V (in vitro) for GFP. Flow cytometry data were analyzed in R using packages flowCore v1.52.1 (Hahne, F. et al., BMC Bioinformatics 10, 106 (2009)), Phenoflow v1.1.2 (Props, R., et al., Methods Ecol. Evol. 7, 1376-1385 (2016)), and ggcyto v1.14.0 (Van, P., et al., Bioinformatics 34, 3951-3953 (2018)). Typically, between 10,000 and 100,000 events were collected per sample, and data were rarefied after gating on FSC and SSC. Background events were accounted for on a per-mouse basis. For co-colonization with the sfgfp-marked and mcherry-marked strains, GFP+ and mCherry+ events from Day −3 (pre-E. coli) were used to subtract background at subsequent timepoints. For colonization with the double-marked strain, GFP+ mCherry+ events from Day −5 (pre-E. coli) were used to subtract background of double fluorescence at subsequent timepoints, and GFP− mCherry+ events from Day 0 (pre-phage) were used to subtract background of red fluorescence at subsequent timepoints. For exclusion of timepoints due to lack of colonization, the background threshold was calculated as the maximum background observed for that population across all timepoints multiplied by a factor of three.

Extraction and analysis of plasmid DNA from in vitro or in vivo isolates. Isolates were cultured on LB or Difco MacConkey agar plates supplemented with the appropriate antibiotics. Colonies were picked, streak-purified, and inoculated into LB or TB supplemented with the appropriate antibiotics. Plasmid DNA was extracted using a QIAprep Spin Miniprep Kit (Qiagen 27106), eluted in TE buffer, and incubated at 60° C. for 10 min. Samples were quantified using a NanoDrop One spectrophotometer and 200-600 ng was digested with FastDigest restriction enzymes (KpnI, Thermo Scientific FD0524; XbaI, Thermo Scientific FD0684) for 10 min at 37° C. followed by gel electrophoresis. Spacer sequences on phagemids were confirmed by Sanger sequencing using primer PSP108.

Quick extraction and PCR analysis of genomic DNA from in vitro or in vivo isolates. Genomic DNA was extracted crudely to use as template for PCR. Briefly, 1.5 ml to 3 ml of culture was transferred to a microfuge tube, cells were pelleted by centrifuging, and the supernatant was discarded. The pellet was frozen, allowed to thaw on ice, resuspended in 100 μl TE, and incubated at 100° C. for 15 min in an Eppendorf ThermoMixer. Samples were cooled on ice, cell debris was pelleted by centrifuging at 21,000 g for 1 min, the supernatant was transferred to a new tube, and diluted 1-in-100 in TE to use as template DNA. PCR was performed using KOD Hot Start DNA polymerase (Millipore 71842-3) using primers KL207/KL200 for the sfgfp gene and primers BAC338F/BAC805R for the 16S rRNA gene (Yu, Y., et al., Biotechnol. Bioeng. 89, 670-679 (2005)).

Analysis of fecal isolates post-delivery of pBluescript II phagemid. Isolates were obtained by streaking fecal suspensions onto LB agar supplemented with carbenicillin followed by streak purification. Single colonies were inoculated in LB supplemented with carbenicillin and streptomycin. Plasmid DNA was extracted for restriction enzyme digest (see above) and genomic DNA was extracted using a DNeasy Blood & Tissue Kit (Qiagen 69506) for Illumina sequencing. The parent strain used to colonize the mice (KL90; SmR W1655 F+) was sequenced as a negative control. Sequence reads were quality filtered using fastp (Chen et al.) and bowtie2 (Langmead, B. & Salzberg, S. L., Nat. Methods 9, 357-359 (2012)) was used to align reads simultaneously to the pBluescript II plasmid sequence (NCBI accession X52329.1) and the complete genome of strain KL68 (W1655 F+) obtained by Nanopore/Illumina sequencing and hybrid assembly.

Extraction of DNA for hybrid assembly. E. coli strains KL68 (W1655 F+ or ATCC 23590), KL114 (W1655 F+ rpsL-Sm^{R} sfgfp), and KL204 (W1655 F+ rpsL-Sm^{R} sfgfp mcherry) were cultured in 50 ml LB supplemented with streptomycin. Cells were collected by centrifuging at 6,000 g for 10 min at room temperature, washed in 10 ml 10 mM Tris 25 mM EDTA (pH 8.0), and resuspended in 4 ml of the same buffer. 12.5 mg lysozyme (Sigma-Aldrich L6876), 100 μl 5 M NaCl, and 50 μl 10 mg/ml RNase A (Thermo-Fisher EN0531) were added and the mixture was incubated at 37° C. for 15 min. To lyse cells, 350 μl 5 M NaCl, 20 μl 20 mg/ml Proteinase K (Ambion AM2546), and 500 μl 10% SDS were added, and the mixture was incubated at 60° C. for 1 h with gentle inversions. 2.75 ml of 7.5 M ammonium acetate was added, and the mixture was incubated on ice 20 min to precipitate proteins. Debris was removed by centrifuging 20,000 g for 10 min and the supernatant was transferred to a new tube. To extract, an equal volume of chloroform was added and mixed; phases were separated by centrifuging at 2,000 g for 10 min, and the aqueous phase was transferred to a new tube. To precipitate the DNA, 1 volume of isopropanol was added, and the tube was inverted until a white precipitate formed. The DNA was pelleted by centrifuging at 2,000 g for 10 min and the supernatant was removed. The pellet was washed with 500 μl ice-cold 70% ethanol, allowed to dry, 1 ml TE was added, and the pellet allowed to dissolve overnight at To further remove RNA, 250 μl of the genomic prep was transferred to a new tube, 12.5 μl 10 mg/ml RNase A was added, and the mixture was incubated at 37° C. for 2 h with mixing every 30 min. To precipitate the DNA, 0.1 volume of 3 M sodium acetate was added followed by 3 volumes of 100% ethanol, and the mixture was inverted until a white precipitate formed. DNA was pelleted by centrifuging at 2,000 g for 10 min, the supernatant was removed, the pellet washed with 100 μl 70% ethanol, allowed to dry, and resuspended in 100 μl TE. Samples were quantified by Qubit dsDNA BR Assay and DNA integrity was confirmed by 0.4% agarose gel electrophoresis using GeneRuler High Range DNA Ladder (Thermo-Fisher FERSM1353). DNA was used for both Oxford Nanopore sequencing and Illumina sequencing.

Illumina whole genome sequencing. DNA concentration was quantified using PicoGreen (ThermoFisher). Genomic DNA was normalized to 0.18 ng/μl for library preparation. Nextera XT libraries were constructed in 384-well plates using a custom, miniaturized version of the standard Nextera XT protocol. Small volume liquid handlers such as the Mosquito HTS (TTP LabTech) and Mantis (Formulatrix) were used to aliquot precise reagent volumes of <1.2 μl to generate a total of 4 μl per library. Libraries were normalized and 1.2 μl of each normalized library was pooled and sequenced on the Illumina NextSeq or MiSeq platform using 2×146 bp configurations. 12 bp unique dual indices were used to avoid index hopping, a phenomenon known to occur on ExAmp based Illumina technologies.

Oxford Nanopore sequencing and hybrid assembly. PCR-free long read libraries were prepared using the Ligation Sequencing Kit (SQK-LSK109), multiplexed using the Native Barcoding Kit (EXP-NBD114), and sequenced on the MinION platform using flow cell version MINT106 (Oxford Nanopore Technologies). Basecalling of MinION raw signals was done using Guppy (v2.2.2, Oxford Nanopore Technologies). Reads were demultiplexed with qcat (v1.1.0, Oxford Nanopore Technologies). Quality control was achieved using porechop (v0.2.3 seqan2.1.1) (https://github.com/rrwick/Porechop) using the discard middle option. Reads were filtered using NanoFilt (v2.6.0) (De Coster, W., et al., Bioinformatics 34, 2666-2669 (2018)) with the following parameters: minimum average read quality score of 10 (−q 10) and minimum read length of 100 (−1 100). Illumina reads were quality filtered using fastp (v0.20.1) (Chen et al.) with the following parameters: cut front, cut tail, cut window size 4, cut mean quality 20, length required 60. Filtered MinION and Illumina reads were then provided to Unicycler (v0.4.8) (Wick, R. R., et al., PLoS Comput. Biol. 13, e1005595 (2017)) for hybrid assembly; default parameters were used unless otherwise noted.

Analysis of isolates after in vitro or in vivo delivery of CRISPR-Cas9 phagemid. Isolates from in vitro GFP-targeting experiments were streak purified 4 times on LB agar supplemented with carbenicillin to ensure clonality. Isolates from in vivo experiments were obtained by suspending a fecal pellet in 500 μl PBS, streaking the suspension onto LB agar supplemented with carbenicillin and streptomycin, followed by streak purification of single colonies. All isolates were cultured in 3 ml TB supplemented with streptomycin and carbenicillin. Cells were pelleted and resuspended in 460 ul of freshly prepared buffer [per sample: 400 ul 10 mM Tris (pH 8.0) 25 mM ETDA, 50 μl 5 M NaCl, and 10 μl 10 mg/ml RNase A (Thermo-Fisher EN0531)]. 50 μl 10% SDS were added, mixed well, and samples were incubated at 60° C. for 1 h with periodic inversions. 260 μl of 7.5 M ammonium acetate was added, and the mixture was incubated on ice 20 min to precipitate proteins. Precipitate was removed by centrifuging 21,000 g for 5 min and the supernatant was transferred to a new tube. To extract, an equal volume of chloroform was added and mixed; phases were separated by centrifuging at 21,000 g for 2.5 min. The aqueous phase was transferred to a new tube, centrifuged at 21,000 g for 2.5 min, and 500 ul was transferred to a new tube. To precipitate the DNA, 500 μl isopropanol was added, and the tube was inverted until a white precipitate formed. Using a pipette tip, the clump was transferred to a new tube, washed with 100 μl cold 70% ethanol, and allowed to dry. 50 μl TE was added and the pellet was allowed to dissolve at 4° C. overnight. DNA integrity was confirmed by gel electrophoresis and used for Illumina whole genome sequencing (see above). Sequence reads were quality filtered using fastp (Chen et al.) and compared against the complete genome of the strain used for in vitro (KL114; sfgfp) or in vivo (KL204; sfgfp mcherry) experiments; reference genomes were generated using Nanopore/Illumina hybrid assembly (see above). To assess deletion size, breseq (Deatherage, D. E. & Barrick, J. E., Methods Mol. Biol. 1151, 165-188 (2014)) was used. To visualize read depth, bowtie2 was used to align reads to the reference, samtools (Li, H. et al., Bioinformatics 25, 2078-2079 (2009)) to filter multi-mapping and low-quality read alignments with MAPQ<2 (view −q 2), and depth was visualized using a sliding window of 20.

### Example 1

### Bacteriophage M13 Enables the Delivery of DNA to the Gut Microbiome

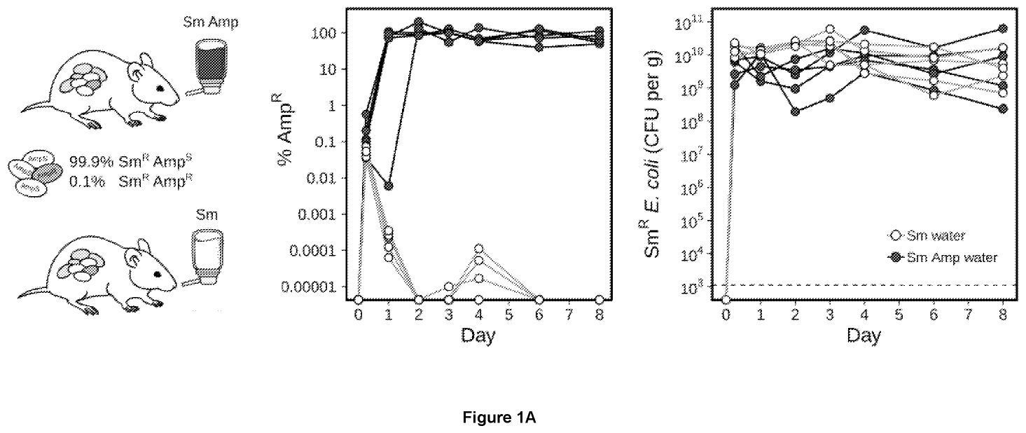

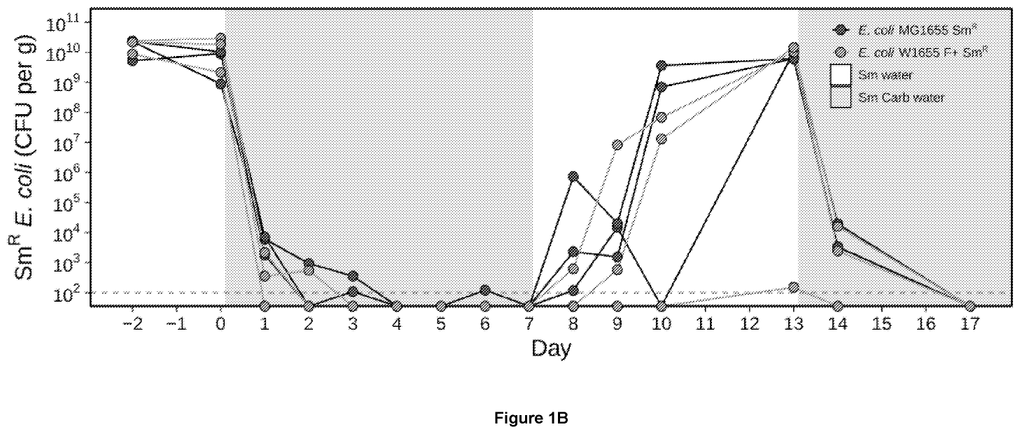

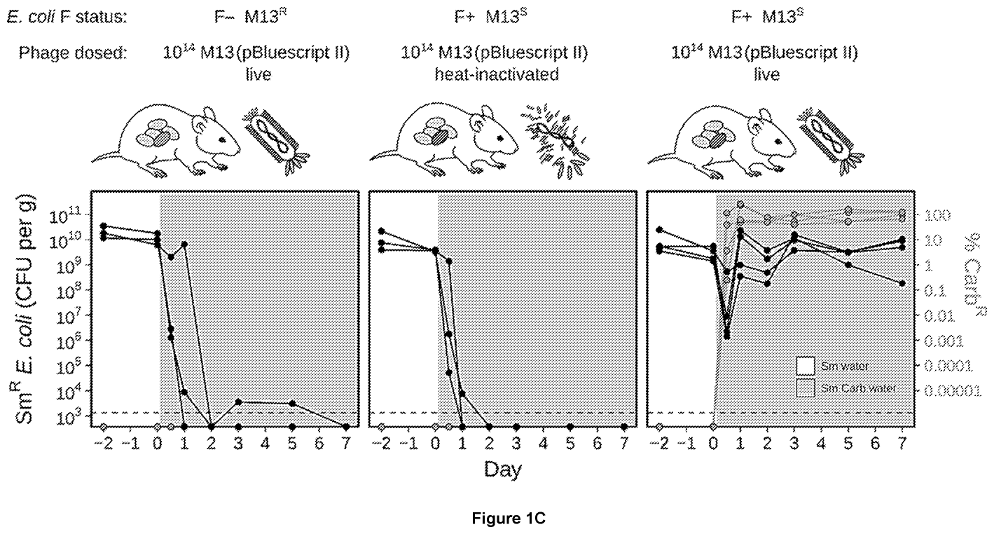

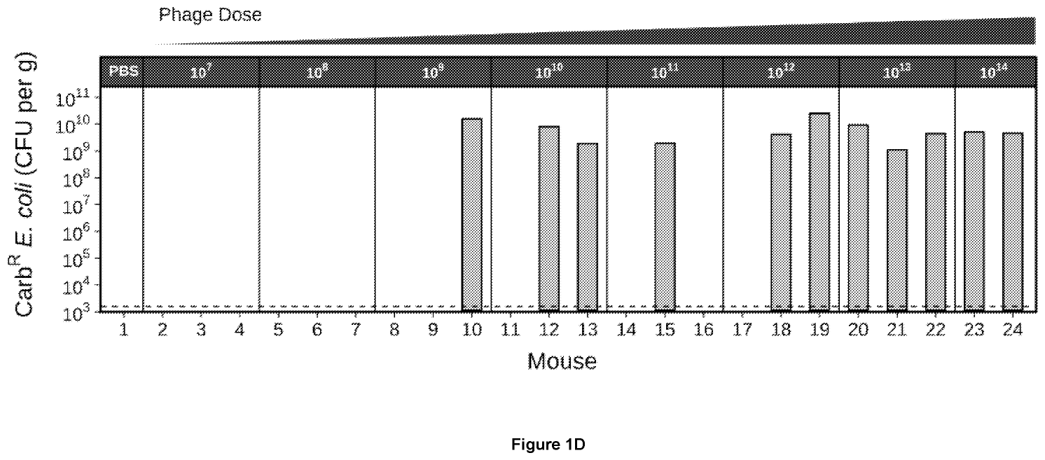

Phagemid pBluescript II (Alting-Mees, M. A. & Short, J. M. pBluescript II: gene mapping vectors. Nucleic Acids Res. 17, 9494 (1989)) carrying the bla (β-lactamase) gene and a β-lactam antibiotic was used in the drinking water to select for successfully infected E. coli. pBluescript II conferred in vitro resistance to ampicillin and the semi-synthetic analogue carbenicillin at concentrations exceeding 1 mg/ml. Sm-treated mice were used to engraft streptomycin-resistant (Sm^{R}) E. coli in the GI tract. As expected, streptomycin altered gut microbial community structure while decreasing diversity and colonization level. Sm^{R} E. coli engrafted at a high proportion (median 18% of the gut microbiota; range 1.4-43%) four days after gavage. A Sm^{R} E. coli population was introduced that was a mixture of 99.9% Amp^{S} (no plasmid) and 0.1% Amp^{R} cells (pBluescript II), split the mice into two groups with access to water containing only streptomycin or both streptomycin and ampicillin, and tracked both total E. coli and Amp^{R} E. coli in mouse feces. At 6 hours post-E. coli introduction, the percentage of Amp^{R} E. coli in the feces of all mice was at or close to 0.1%, consistent with the gavaged mixture transiting through the GI tract. Within 1-2 days, mice on water containing ampicillin exhibited an increase in the percent of Amp^{R} E. coli by 3 orders of magnitude, reaching complete or near complete colonization (FIG. 1a). In contrast, the Amp^{R} subpopulation was lost in mice on water without ampicillin. These results demonstrate β-lactam antibiotics can be used to select for resistant E. coli.

Antibiotics were capable of eradicating a sensitive population of E. coli that had established stable colonization in the mouse gut. Sm-treated mice were engrafted with Sm^{R} E. coli MG1655 or W1655 F+ and tracked colonization levels during treatment with the β-lactam antibiotic carbenicillin. Carbenicillin decreased the median E. coli colonization level from 9.6×10⁹ to 2.0×10³ CFU/gram feces in the first day, and levels decreased to below our limit of detection (˜10² CFU/g) in all mice over the course of treatment (FIG. 1b). When selection was lifted on Day 7, recolonization was observed for 5/6 mice; when carbenicillin was reintroduced on Day 13, colonization again dropped dramatically. The low background of E. coli in the gut during carbenicillin treatment, as well as the lack of spontaneous resistant cells able to reestablish, supports the utility of this model for assessing the phage-mediated delivery of a resistance gene.

Next, the ability to deliver an antibiotic resistance gene to E. coli within the gut was tested. Sm-treated mice were colonized with either Sm^{R} E. coli W1655 F+(M13^{S}) or W1655 F− (M13^{R} as a control), and dosed the each animal with either live or heat-inactivated M13 carrying pBluescript II (FIG. 1c). After dosing the mice with 10¹⁴ M13(pBluescript II), they were immediately transferred to water containing carbenicillin and tracked both total E. coli and Carb^{R} E. coli in the feces. E. coli colonization fell rapidly and stayed near or below the limit of detection in control mice that were either colonized with F— and given live phage or colonized with F+ but given heat-inactivated phage. In contrast, mice colonized with F+ and dosed with live phage had a transient drop in colonization on the first day, during which the rise of Carb^{R} cells occurred, and colonization was re-established within one day by an E. coli population resistant to carbenicillin (FIG. 1c). These results suggest that orally dosed M13 phage were able to infect E. coli in the gut and deliver a plasmid conferring resistance to carbenicillin.

M13-mediated pBluescript II delivery to E. coli in the gut was replicated in an independent animal experiment. Sm-treated mice were colonized by Sm^{R} E. coli W1655 F+ and orally dosed with ten-fold serial dilutions of M13(pBluescript II). Colonization by Carb^{R} E. coli was consistent at high doses but variable at lower doses indicating that the probability of successful colonization increases with phage dose (FIG. 1d; P=0.009, odds ratio=2.5, logistic regression). Plasmid DNA of the expected size was detected in fecal Carb^{R} E. coli isolates from all 11 mice that were successfully colonized. Genome sequencing confirmed the presence of pBluescript II in these 11 isolates, which was undetectable in the parent strain. These results indicate that plasmid DNA was transferred from M13 phage into recipient E. coli colonizing the GI tract.

Finally, this experiment was repeated in the absence of carbenicillin selection. Sm^{R} E. coli was engrafted in mice, each mouse was gavaged with M13(pBluescript II), and tracked both infected (Carb^{R}) and total (Sm^{R}) E. coli in feces. The fraction of phage-infected Carb^{R} E. coli was low, reaching a maximum of 0.1% of the total population, potentially indicative of poor phage survival during GI transit. Mice were gavaged with M13(pBluescript II) and assayed for viable phage in the feces. The median output of viable M13(pBluescript II) was reduced to 1×10⁶ relative to an input of 6×10¹³. M13(pBluescript II) is acid tolerant in vitro, suggesting that additional factors may be responsible for the low in vivo viability and emphasizing the benefits of pairing gene delivery with antibiotic selection.

### Example 2

### M13 Carrying CRISPR-Cas9 can Target E. coli In Vitro

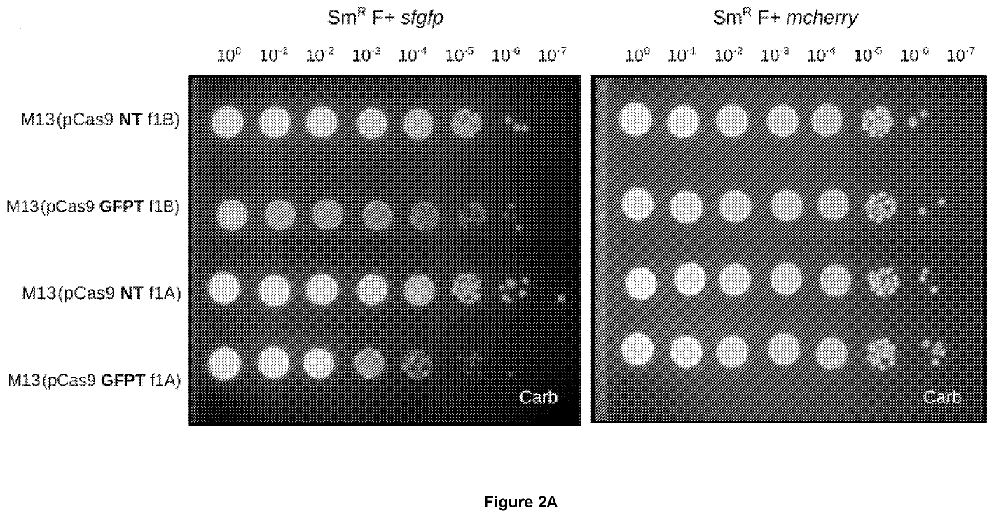

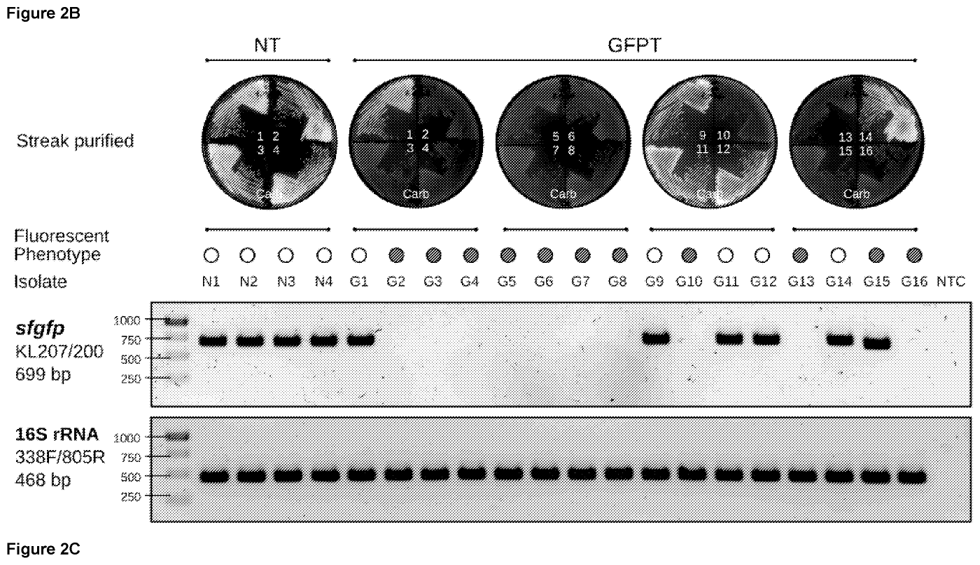

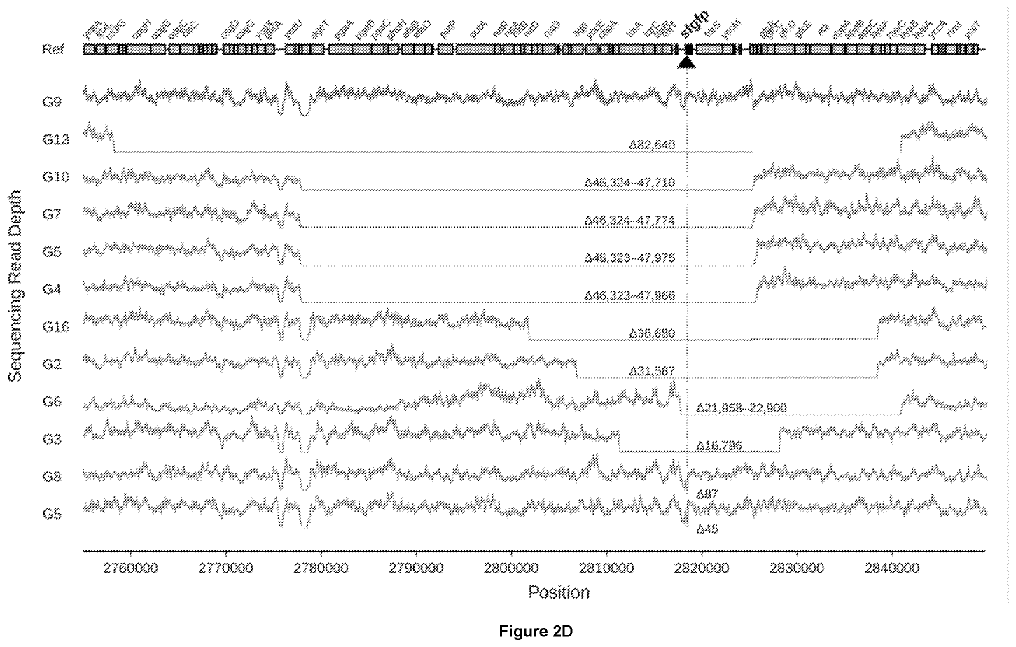

Two fluorescently marked isogenic derivatives of Sm^{R} W1655 F+ were generated using the mcherry (red fluorescence) or the sfgfp (green fluorescence) marker gene. Next, M13-compatible non-targeting (NT) and GFP-targeting (GFPT) CRISPR-Cas9 vectors were constructed by cloning the spacers sequences, bla gene, and f1 origin of replication into the previously described low-copy vector pCas9²⁰, generating pCas9-NT-f1A/B and pCas9-GFPT-f1A/B. The bla and f1 ori were cloned as a fragment from pBluescript II in both possible orientations (A or B) to make possible M13 ssDNA packaging of either strand of vector DNA. These phagemids were packaged into M13 using a helper strain and called the resulting phage NT-M13 or GFPT-M13. The two phage were used to infect the GFP+ or mCherry+ strains and cells were diluted and spotted on solid media containing carbenicillin to select for the transferred phagemid. GFP+ E. coli infected with GFPT-M13 exhibited impaired colony growth relative to the NT-M13 control (FIG. 2a). Total CFUs were not markedly affected, indicating that cells can recover from M13-delivered CRISPR-Cas9 targeting.

Analysis of the surviving cells provided mechanistic insights. Colonies arising from infection with NT-M13 or GFPT-M13 were streak purified, allowing us to pick a mixture of bright and dim colonies. Of 16 GFPT clones analyzed, 11 were non-fluorescent (FIG. 2b). PCR amplification of sfgfp confirmed the intact gene in 4 NT controls and all 5 GFPT clones that retained fluorescence (FIG. 2c). Sanger sequencing revealed that 1 GFPT clone had a point mutation in the sfgfp target while the 4 others had lost the spacer in the CRISPR-Cas9 phagemid that leads to targeting. All 11 non-fluorescent GFPT clones retained the spacer and had chromosomal deletions at the target locus: 10 were PCR negative (FIGS. 2c) and 1 had a small deletion within sfgfp. Finally, whole genome sequencing was used to define the size of each deletion, which ranged from 45 bp to 82.6 kb (FIG. 2d), consistent with prior work demonstrating that E. coli can repair Cas9-induced double-stranded breaks through homologous recombination (Cui, L. & Bikard, D., Nucleic Acids Res., 44, 4243-4251 (2016)).

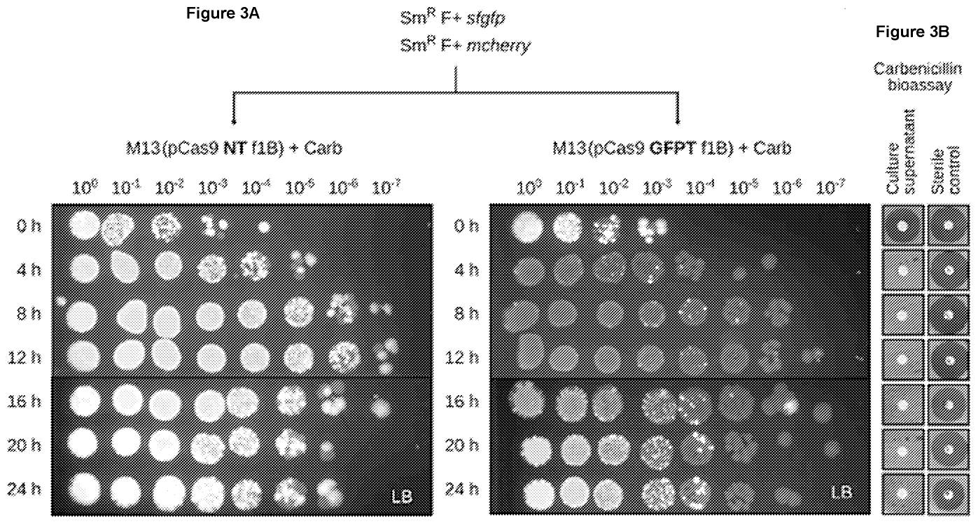

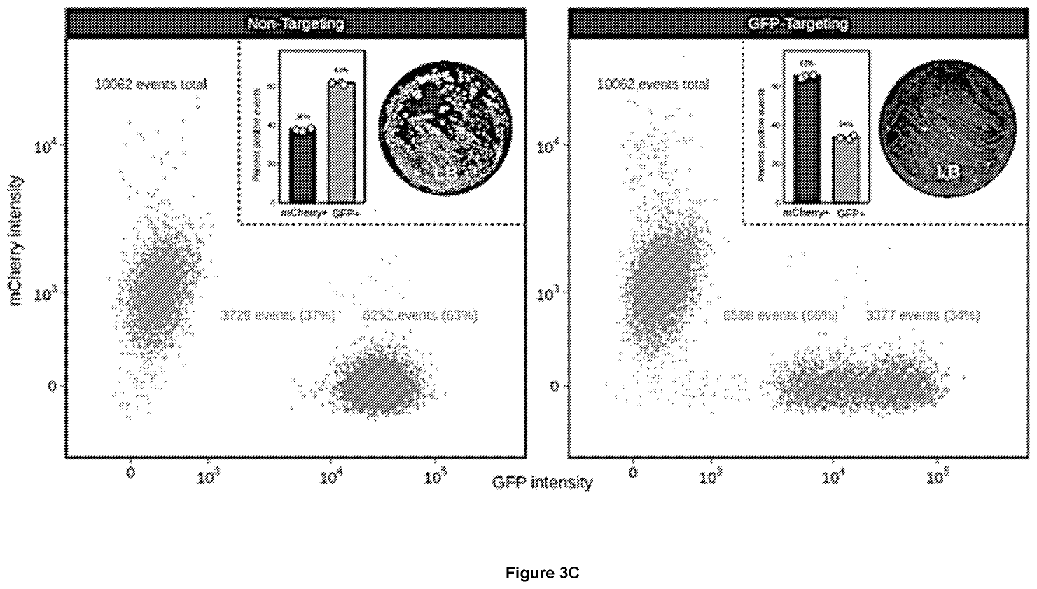

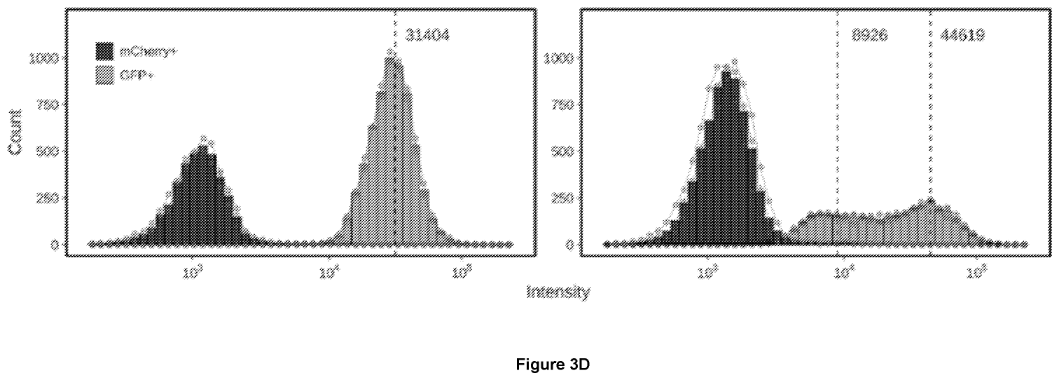

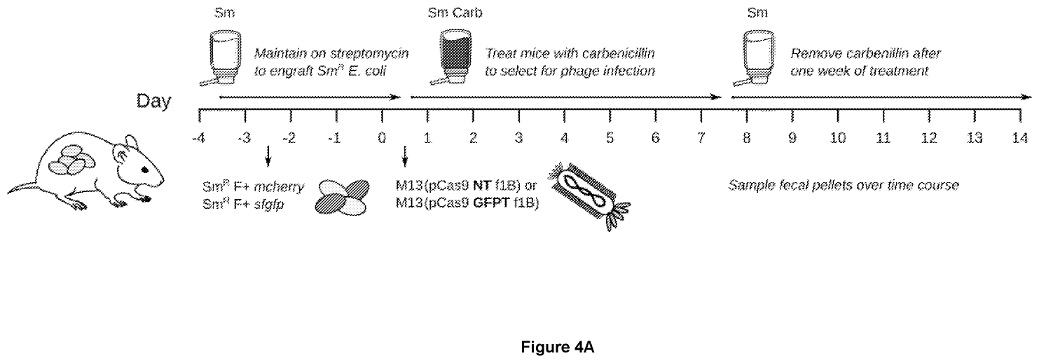

These results led to the hypothesis that targeted cells would be less able to recover during competitive growth. GFP+ and mCherry+ E. coli, were co-cultured, adding either NT-M13 or GFPT-M13 followed by carbenicillin to select for phage infection. GFPT-M13 decreased the frequency of GFP+ colonies by 4 hours, relative to the NT-M13 control (FIG. 3a). At later time points (16-24 hours), healthy GFP+ colonies increased in abundance, consistent with low levels of carbenicillin after 4 hours in cultures expressing the β-lactamase resistance gene (FIG. 3b). The loss of selection for the phagemid was confirmed by re-analyzing our colonies on selective media. Next, flow cytometry was used to better quantify the two strains in an independent experiment. Compared to the NT-M13 control, GFPT-M13 co-cultures exhibited fewer GFP+ events (FIG. 3c) and a bimodal distribution of fluorescence (FIG. 3d). Counts of GFP+ cells were higher by flow cytometry than on solid media for the same co-cultures (FIG. 3c inset), consistent with an impaired growth of these cells. GFP+ events further decreased at 24 hrs in the GFPT-M13 group. Taken together, these results suggest that competitive growth can increase the efficiency of targeting a strain for depletion due to the resulting growth impairments in the targeted strain.

### Example 3

### Sequence-Specific Depletion of E. coli within the Mouse Gut Microbiota

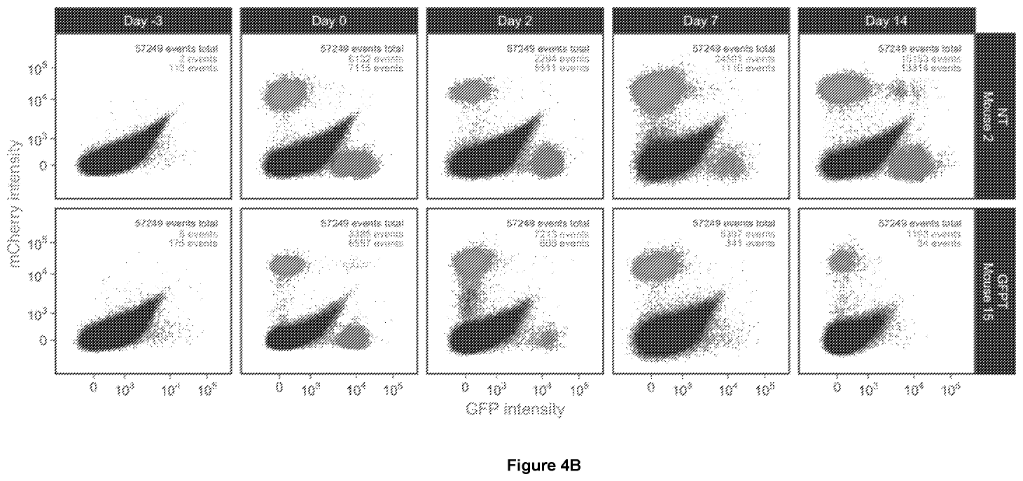

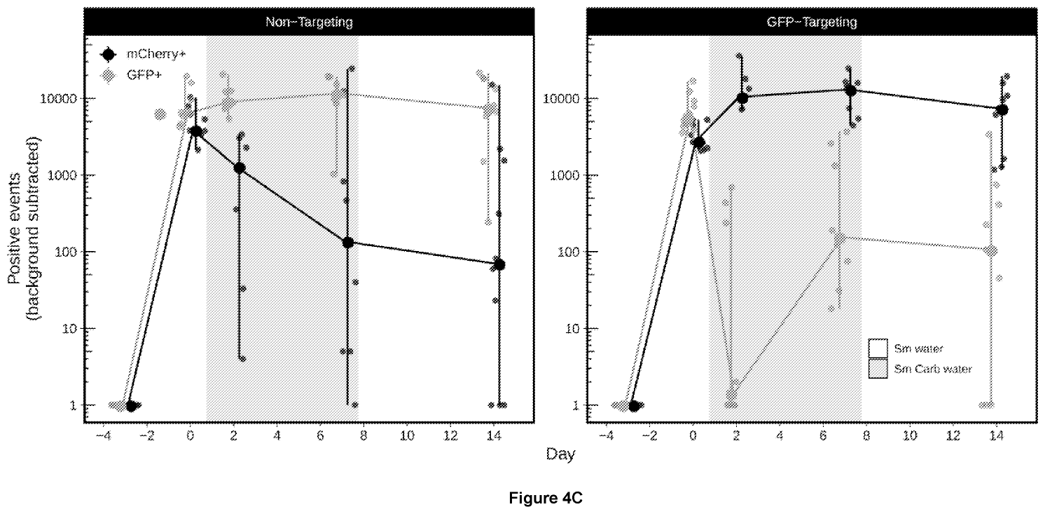

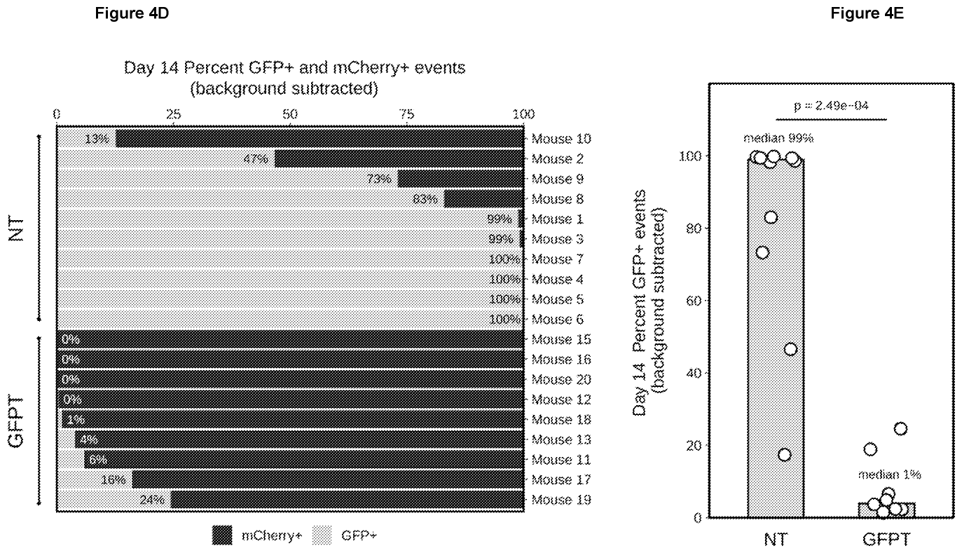

Sm-treated mice were co-colonized with both Sm^{R} F+ sfgfp and Sm^{R} F+ mcherry strains, orally dosed with either 10¹¹ NT-M13 or GFPT-M13, and carbenicillin was added in the water to select for phage infection. After one week of treatment, carbenicillin was removed from the water and followed mice for an additional week to determine whether phage-induced changes would persist in the absence of maintaining selection (FIG. 4a). Flow cytometry revealed that the GFP+ strain outcompeted the mCherry+ strain in the NT-M13 group (FIGS. 4b,c). In contrast, GFP+ events in the GFPT-M13 group exhibited a sharp decrease on Day 2, followed by a recovery on Days 7 and 14 to levels below the NT-M13 group (FIGS. 4b,c). Culturing from mouse stool confirmed the decreased GFP+ events on Day 2. In 4 mice that received GFPT-M13, the mCherry+ strain fixed in the population (GFP+ events were below background), an outcome that was not observed for any mouse in the NT-M13 group (FIG. 4d). Despite lifting the carbenicillin selection for 1 week, endpoint GFP+ events remained significantly lower in the GFPT-M13 group relative to NT-M13 controls (FIG. 4e; P=0.0002, Mann-Whitney test). These data support the utility of M13-delivered CRISPR-Cas9 for sequence-specific depletion of an otherwise isogenic bacterial strain in the mouse gut.

### Example 4

### M13-Delivered CRISPR-Cas9 Induces Chromosomal Deletions in the Gut Microbiome

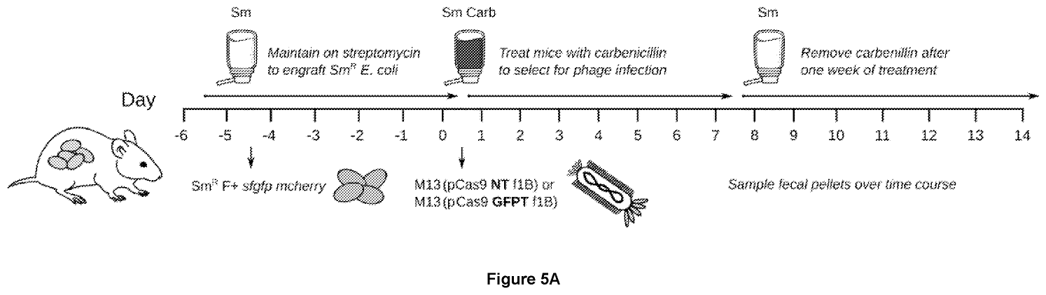

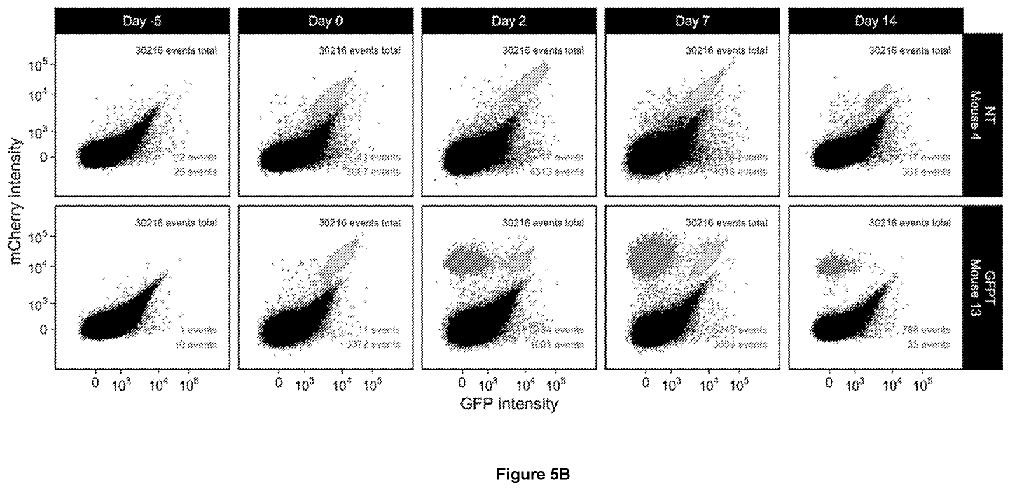

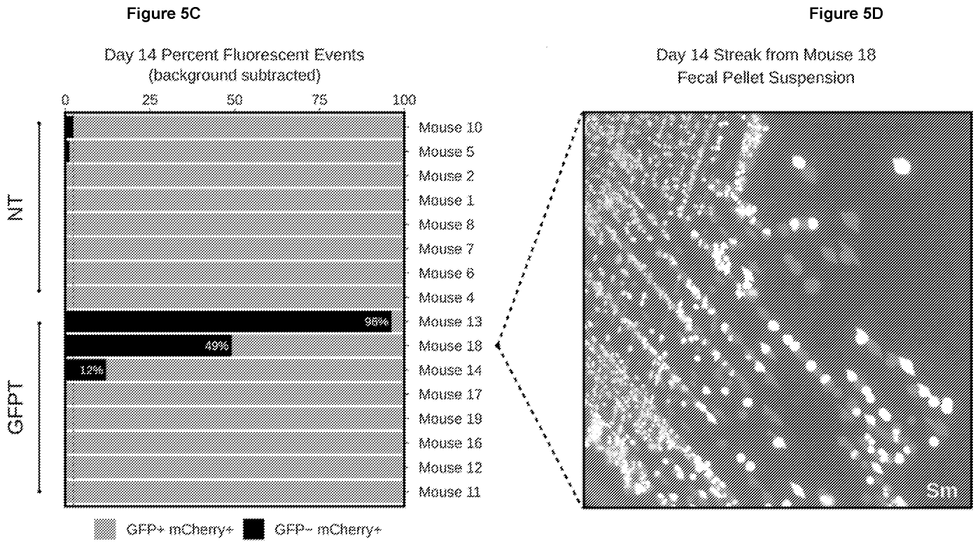

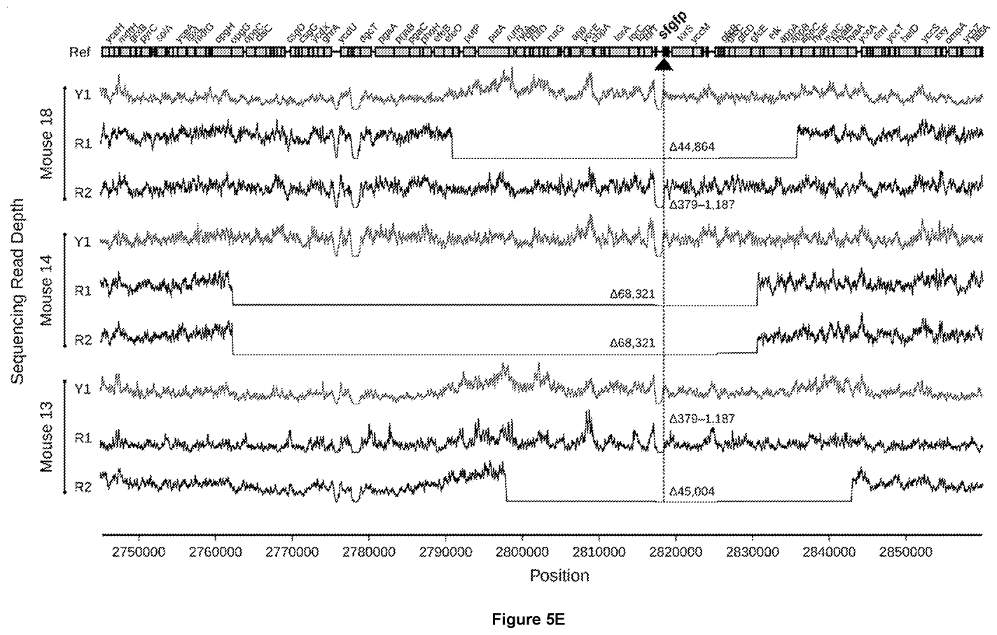

A double-marked Sm^{R} F+ sfgfp mcherry strain was constructed to quantify the efficiency of gene deletion. This strain was introduced into Sm-treated mice, orally dosed each mouse with either 10¹¹ NT-M13 or GFPT-M13, and added carbenicillin in the water; after one week, carbenicillin was removed and the mice were followed for another week (FIG. 5a). GFP-mCherry+events were detectable in GFPT-M13 but not NT-M13 mice, indicative of successful CRISPR-Cas9 delivery and gene deletion (FIG. 5b). By the final timepoint, GFP− mCherry+ events were detected in 3/8 mice (FIG. 5c). The relative abundance of GFP− mCherry+ cells varied from 12-96% (FIG. 5c). Culturing on solid media confirmed the presence of viable red fluorescent colonies in proportions consistent with flow cytometry results (FIG. 5d).

To more definitively assess the presence or absence of the targeted genomic locus and the CRISPR-Cas system, GFP− mCherry+ and GFP+ mCherry+ E. coli were isolated from Day 2 mouse stool. All of the GFP+ mCherry+ isolates from the NT-M13 group and the GFP-mCherry+ isolates from the GFPT-M13 group had an intact spacer sequence. In contrast, 4/5 GFP+ mCherry+ isolates from the GFPT-M13 group had lost the spacer. Of note, the remaining isolate lost cas9, and parts of the CRISPR array and tracrRNA. Whole genome sequencing was used to confirm putative chromosomal deletions and to quantify their size. Two representative colonies were analyzed from each of the 3 mice with detectable GFP− mCherry+ cells, revealing a wide range in deletion sizes that were not observed in a control GFP+ mCherry+ isolate from each animal (FIG. 5e). These results indicate that while it is possible for CRISPR-Cas9-induced genomic deletion events to occur in vivo, resultant deletion strains may or may not outcompete the parent strain due to the potential to evade targeting through loss of some or all of the exogenous CRISPR-Cas system.

The aforementioned Examples and results provide a proof-of-principle for strain-specific targeting within the GI tract, the full eradication of the targeted strain was difficult to achieve due to the ability of bacterial cells to survive Cas9-induced double-stranded breaks by homologous recombination (Gynnå, A. H., et al., bioRxiv (2020) doi:10.1101/2020.02.13.946996) or non-homologous end-joining mechanisms (Cui, L. & Bikard, D., Nucleic Acids Res., 44, 4243-4251 (2016).) CRISPR-Cas9 may be better suited to induce targeted genomic deletions, leveraging the conserved DNA repair pathways present in bacteria. An advantage of this approach is that the deletion of a single genomic locus is unlikely to have as large an impact on the rest of the gut microbiota than if the strain were to be removed entirely, particularly for “keystone” species that serve unique functional roles. Remarkably, a wide range of deletion sizes (379-68,321 bp) were detected, highlighting the ability of bacteria to survive large deletions and opening up the potential for the in situ removal of entire biosynthetic gene clusters or pathogenicity islands. In turn, the data provided herein suggests that it may also be feasible to deliver more complex genetic circuits to E. coli, with the goal of boosting metabolic pathways beneficial to its mammalian host.

The present disclosure thus provides a robust and modular toolkit for microbiome editing.

The various embodiments described above can be combined to provide further embodiments. All U.S. patents, U.S. patent application publications, U.S. patent application, foreign patents, foreign patent application and non-patent publications referred to in this specification and/or listed in the Application Data Sheet are incorporated herein by reference, in their entirety. Aspects of the embodiments can be modified if necessary to employ concepts of the various patents, applications, and publications to provide yet further embodiments.

These and other changes can be made to the embodiments in light of the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

What is claimed is:

1. A method of selectively engineering at least one bacterial strain among a mixed population of bacterial strains in the gut of a subject comprising administering at least one bacteriophage comprising at least one nucleic acid, wherein said bacteriophage selectively infects the at least one bacterial strain under conditions that allow expression of said at least one nucleic acid.

2. The method of claim 1 wherein the at least one bacterial strain is a member of a species selected from the group consisting of Escherichia coli, Escherichia albertii, Klebsiella pneumoniae, and Salmonella typhimurium.

3. The method of claim 1 wherein the at least one bacterial strain is a member of a genus selected from the group consisting of Escherichia, Salmonella, Shigella, and Klebsiella

4. The method of claim 1 wherein the at least one bacterial strain is a member of Enterobacteriaceae.

5. The method of claim 1 wherein 1, 2, 3, 4, 5, 6, 7, 8, 9, 10 or more bacterial strains are selectively engineered by the same bacteriophage.

6. The method of any of the preceding claims wherein the subject is a mammal.

7. The method of claim 4 wherein the mammal is a human.

8. The method of any of the preceding claims wherein the at least one bacteriophage is selected from the group consisting of M13, T7, T4, lambda, T3, T1, P1 and Mu, and derivatives thereof.

9. The method of claim 8 wherein 1, 2, 3, 4, 5 or more bacteriophage are administered.

10. The method of any of the preceding claims wherein the nucleic acid is a phagemid, a cosmid, and a phage-plasmid hybrid vector suitable for use with said at least one bacteriophage.

11. The method of claim 10 wherein the nucleic acid comprises a phage-plasmid hybrid vector.

12. The method of claim 11 wherein the phage-plasmid hybrid vector comprises one or more genes encoding a protein, an enzyme, or an RNA.

13. The method of claim 12 wherein the one or more genes is an antibiotic resistance gene.

14. The method of claim 12 wherein the one or more genes encode an RNA-guided nuclease.

15. The method of claim 14 wherein the RNA-guided nuclease is Cas9.

16. The method of claim 15 wherein the Cas9 specifically targets an antibiotic resistance gene in the at least one bacterial species.

17. The method of claim 10 wherein the one or more genes encodes an enzyme selected from the group consisting of: an enzyme for drug activation, an enzyme for drug detoxification, an enzyme for transformation of dietary components into beneficial compounds for the host, an enzyme involved in one or more biosynthetic pathways for de novo production of beneficial compounds for the host (e.g. anti-inflammatory), an enzyme to aid long-term engraftment of the strain in the host gut, and an enzyme to increase competitive advantage of the strain in the host gut relative to the parent strain before gene delivery.

18. A method of treating a disease associated with at least one bacterial strain among a mixed population of bacterial strains in the gut of a subject comprising administering at least one bacteriophage to said subject comprising at least one nucleic acid, wherein said bacteriophage selectively infects the at least one bacterial strain under conditions that allow expression of said at least one nucleic acid, wherein said at least one nucleic acid comprises one or more genes.

19. A method of increasing the growth of one or more target strain or species or genus in the gut microbiota of a subject comprising administering at least one bacteriophage to said subject comprising at least one nucleic acid, wherein said bacteriophage selectively infects the at least one bacterial strain under conditions that allow expression of said at least one nucleic acid, wherein said at least one nucleic acid comprises one or more genes.

20. A method of eliminating or reducing the population of one or more target strain or species or genus from an established community in the gut microbiota of a subject comprising administering at least one bacteriophage to said subject comprising at least one nucleic acid, wherein said bacteriophage selectively infects the at least one bacterial strain or species or genus under conditions that allow expression of said at least one nucleic acid, wherein said at least one nucleic acid comprises one or more genes.

21. A method of conferring antibiotic resistance to one or more strain or species or genus of bacteria in an established community in the gut microbiota of a subject comprising administering at least one bacteriophage to said subject comprising at least one nucleic acid, wherein said bacteriophage selectively infects the at least one bacterial strain or species or genus under conditions that allow expression of said at least one nucleic acid, wherein said at least one nucleic acid comprises one or more genes that confer antibiotic resistance.

22. A method of modifying the genome of one or more strain or species or genus of bacteria in an established community in the gut microbiota of a subject comprising administering at least one bacteriophage to said subject comprising at least one nucleic acid, wherein said bacteriophage selectively infects the at least one bacterial strain or species or genus under conditions that allow expression of said at least one nucleic acid, wherein said at least one nucleic acid comprises one or more genes.

23. A method of upregulating or downregulating at least one gene in one or more strain or species or genus of bacteria in an established community in the gut microbiota of a subject comprising administering at least one bacteriophage to said subject comprising at least one nucleic acid, wherein said bacteriophage selectively infects the at least one bacterial strain or species or genus under conditions that allow expression of said at least one nucleic acid, wherein said at least one nucleic acid comprises one or more genes or encodes for a functional RNA molecule capable of upregulating or downregulating the at least one gene.

24. A method of selectively removing at least one bacterial strain or species or genus from a population of bacteria in the gut of a subject comprising administering at least one bacteriophage to said subject comprising at least one nucleic acid, wherein said bacteriophage selectively infects the at least one bacterial strain or species or genus under conditions that allow expression of said at least one nucleic acid, wherein said at least one nucleic acid comprises one or more genes.

25. The method of any one of claims 1-7, 9-12, and 18-24, wherein the bacteriophage is selected from the group consisting of M13, T7, T4, lambda, T3, T1, P1 and Mu, and derivatives thereof, and wherein said at least one nucleic acid encodes one or more sequences or genes involved in RNA-guided genome modification.

26. The method of claim 25, wherein the bacteriophage is M13 and wherein said M13 comprises a nucleotide sequence encoding Cas9.


SEQUENCE LISTING (17 sequences)

SEQ ID NO: 1 - DNA, 20 bp, Artificial Sequence
  Synthetic
cgtggcatggaaatactccg

SEQ ID NO: 2 - DNA, 20 bp, Artificial Sequence
  Synthetic
gcatcgccctaaaattcggc

SEQ ID NO: 3 - DNA, 34 bp, Artificial Sequence
  Synthetic
aaacccttcaccttcaccacgaacagagaatttg

SEQ ID NO: 4 - DNA, 34 bp, Artificial Sequence
  Synthetic
aaaacaaattctctgttcgtggtgaaggtgaagg

SEQ ID NO: 5 - DNA, 34 bp, Artificial Sequence
  Synthetic
aaacatcgcacatcctggtcgcgacattaagagt

SEQ ID NO: 6 - DNA, 34 bp, Artificial Sequence
  Synthetic
aaaaactcttaatgtcgcgaccaggatgtgcgat

SEQ ID NO: 7 - DNA, 22 bp, Artificial Sequence
  Synthetic
ttaataaatgcagtaatacagg

SEQ ID NO: 8 - DNA, 31 bp, Artificial Sequence
  Synthetic
cctgtcgacggtatcgataagcttgatatcg

SEQ ID NO: 9 - DNA, 39 bp, Artificial Sequence
  Synthetic
cctgtcgacgattatcaaaaaggatcttcacctagatcc

SEQ ID NO: 10 - DNA, 20 bp, Artificial Sequence
  Synthetic
ctgttcaccggtgttgttcc

SEQ ID NO: 11 - DNA, 25 bp, Artificial Sequence
  Synthetic
ttatttgtagagttcatccatgccg

SEQ ID NO: 12 - DNA, 17 bp, Artificial Sequence
  Synthetic
actcctacgggaggcag

SEQ ID NO: 13 - DNA, 21 bp, Artificial Sequence
  Synthetic
gactaccagggtatctaatcc

SEQ ID NO: 14 - DNA, 52 bp, Artificial Sequence
  Synthetic
tcgtcggcagcgtcagatgtgtataagagacaggtgccagcmgccgcggtaa

SEQ ID NO: 15 - DNA, 54 bp, Artificial Sequence
  Synthetic
gtctcgtgggctcggagatgtgtataagagacagggactachvgggtwtctaat

SEQ ID NO: 16 - DNA, 51 bp, Artificial Sequence
  Synthetic
  misc_feature (30)..(37): n is a, c, g, or t
aatgatacggcgaccaccgagatctacacnnnnnnnntcgtcggcagcgtc

SEQ ID NO: 17 - DNA, 47 bp, Artificial Sequence
  Synthetic
  misc_feature (25)..(32): n is a, c, g, or t
caagcagaagacggcatacgagatnnnnnnnngtctcgtgggctcgg